(19) United States
(12) Patent Application Publication
(10) Pub. No.: US 2011/0021765 A1
(43) Pub. Date: Jan. 27, 2011

(54) FLUORINATED LANTHANIDE PROBES FOR 19F MAGNETIC RESONANCE APPLICATIONS

(76) Inventors: David Parker, Durham (GB); Padma Kanthi Senanayake, Durham (GB)

Correspondence Address: MANNAVA & KANG, P.C., 11240 WAPLES MILL ROAD, Suite 300, FAIRFAX, VA, 22030, US

(21) Appl. No.: 12/599,609

(22) PCT Filed: Apr. 11, 2008
(86) PCT No.: PCT/GB2008/001284

§ 371 Date: Jul. 15, 2010

(30) Foreign Application Priority Data
May 1, 2007 (GB) 0708411.4

(51) Int. Cl. C07H 15/26; C07F 5/00; C07D 257/02

(52) U.S. Cl. 536/17.4; 540/465; 540/474

(57) ABSTRACT

The invention provides a paramagnetic lanthanide (III) complex comprising a lanthanide (III) ion and a polydentate ligand wherein the polydentate ligand comprises one or more fluorine atoms in which the distance of at least. one of the fluorine atoms to the lanthanide ion is less than 7, said polydentate ligand not being a DPTA bisamide of p-CF₃-aniline.

This invention relates to the use of fluorinated lanthanide probes for ¹⁹F magnetic resonance applications.

### INTRODUCTION

Chemical probes that incorporate fluorine are of much current interest in positron emission tomography (¹⁸F) and magnetic resonance imaging and spectroscopy (MRI and MRS) (M. Rudin and R. Weissleder, Nature Rev., 2003, 2, 123; M. J. Adam and D. S. Willbur, Chem. Soc. Rev., 2005, 34, 153; O. Couturier, A. Luxen, J. F. Chatal, J. P. Vuillez, P. Rigo and R. Hustinx, Eur. J. Nucl. Med. Mol. Imaging, 2004, 31, 1182; J. X. Yu, V. D. Kodibadkar, W. M. Ciu and R. P. Mason, Curr. Med. Chem., 2005, 12, 819). The high NMR sensitivity and large chemical shift range (>300 ppm), accompanied by a near-zero endogenous background make ¹⁹F-magnetic resonance studies intrinsically attractive for studies in a wide range of chemical and biological systems.

Several reports have described the use of ¹⁹F magnetic resonance studies in quantitative studies to track in vivo the metabolism of ¹⁹F-labelled drugs, e.g. 5-fluorouracil or gemcitabine (L. D. Stegman, A. Rehemtulla, B. Beattie, E. Kievit, T. S. Lawrence, R. G. Blasberg, J. G. Tjuvajev and B. D. Ross, Proc. Natl. Acad. Sci. USA., 1999, 96, 9821). Perfluorinated compounds have also been used, e.g. as tags that may be conjugated to biomolecules to allow the tracking of the conjugate, as in the monitoring of labelled actin during polymerization or in its interaction with myosin (H. R. Kalbitzer, G. Rohr, E. Nowok, R. S. Goody, W. Kuhn and H. Zimmermann, NMR Biomed., 1992, 5, 347). More recently, CF₃ groups have been introduced into arylgalactopyranosides, allowing the activity of the β-galactosidase enzyme to be followed by ¹⁹F NMR spectroscopy (V. D. Kodibagkar, J. Yu, L. Liu, H. P. Hetherington and R. P. Mason, Magn. Reson. Imaging, 2006, 24, 959; J. Yu, V. D. Kodibagkar, W. Cui and R. P. Mason, Biorg. Med. Chem., 2006, 14, 326). In C Geze, A Vacca et al., Bull. Soc. Chim. Fr., 1996, 133, 267-272 there is described the synthesis, characterization and relaxivity of functionalized aromatic amide DTPA-lanthanide complexes for proton NMR studies. There is no discussion in that paper of any ¹⁹F magnetic resonance applications). Using certain trifluoromethyl arylgalactopyranosides, the feasibility of chemical shift imaging studies has been demonstrated in vitro (ca. 50 mM substrate), monitoring the differing shifts of the fluorinated glycoside and phenolic product (Δδ_{F}≦1 ppm). Thus, a ¹⁹F NMR assay for β-galactosidase activity has been advocated (V. D. Kodibagkar et al., infra), in principle allowing gene expression (e.g. for the lacZ gene) to be tracked.

A limiting feature of ¹⁹F NMR work to date relates to the slow longitudinal relaxation rate (R₁=1/T₁) of the ¹⁹F nucleus, especially in CF₃ (trifluoromethyl) groups where R₁ values are of the order of 0.5 to 1 s⁻¹. This determines the spectral acquisition time, owing to the need to wait for an approximately 3 to 6 second repetition time (approximately 3 to 5 times T₁). As signal/noise ratios vary with the square root of the number of scans acquired, it is desired to modulate the chemical environment of the F nucleus or nuclei to increase R₁.

Notwithstanding the early interest in lanthanide shift reagents, based on fluorinated β-diketonates, there has been very little systematic work reported to date concerning the relaxation and dipolar shift effects of lanthanide ions on ¹⁹F NMR spectral parameters. Indeed, examples of the interpretation of ¹⁹F-NMR shifts of paramagnetic complexes are very rare, but do include some work on fluorine-labelled iron(III) porphyrins (L. Yatsunyk and F. A. Walker, Inorg. Chim. Acta., 2002, 327, 266) as well as a ¹⁹F NMR examination of stereoisomerism of trifluoroethyl esters of macrocyclic lanthanide phosphonates (W. D. Kim, G. E. Kiefer, J. Huskens and A. D. Sherry, Inorg Chem., 1997, 36, 4128-4134). Isolated studies have examined the temperature or pH dependence of ¹⁹F relaxation rates resonances ion-paired (e.g. CF₃SO₃⁻ anions (S. Aime, A. Barge, J. I. Bruce, M. Botta, J. A. K. Howard, J. M. Moloney, D. Parker, A. S. de Sousa and M. Woods, J. Am. Chem. Soc., 1999, 121, 5762; S. Aime, A. Barge, M. Botta, D. Parker and A. S. de Sousa, J. Am. Chem. Soc., 1997, 119, 4767; H. Lee, R. R. Price, G. E. Holburn, C. L. Partain, M. D. Adams and W. P. Cacheris, J. Magn. Reson. Imaging, 1994, 4, 609)) or reversibly bound (e.g. trifluorolactate (E. Terreno, M. Botta, P. Boniforte, C. Bracco, L. Milone, B. Mondino, F. Uggeri and S. Aime, Chem. Eur. J. 2005, 11, 5531)) to a lanthanide centre.

### SUMMARY OF THE INVENTION

We have found that a solution to the problem of low ¹⁹F sensitivity in magnetic resonance experiments is to place the ¹⁹F nucleus close to a paramagnetic lanthanide (Ln) ion, in a kinetically stable complex or conjugate. This leads to much faster R₁ (and R₂) relaxation rates, and allows large increases in 19-F NMR signal intensity to be acquired, for a given spectral acquisition time per unit concentration.

Innumerable lanthanide (mainly gadolinium) complexes have been studied over the past 20 years, typically as contrast agents for proton MRI, that are well tolerated in vivo, typically at doses in the 0.1 to 0.2 mM kg⁻¹ range (P. Caravan, J. J. Ellison, T. J. McMurry and R. B. Lauffer, Chem. Rev., 1999, 99, 2293; R. S. Dickins, C. Crossland, J. A. K. Howard, D. Parker and H. Puschmann, Chem. Rev. 2002, 102, 1977).

For example, we have found that polydentate ligands, which may be acyclic or macrocyclic, comprising one or more fluorine atoms, for example as a difluoromethyl, difluoromethylene or CF₃ group, may be complexed to lanthanide (III) ions so as to increase the R₁ (and R₂) relaxation rates of the ¹⁹F nucleus or nuclei. The invention thus provides paramagnetic lanthanide (III) complexes comprising a lanthanide (III) ion and a polydentate ligand wherein the polydentate ligand comprises one or more fluorine atoms and wherein at least one of the fluorine atoms has a larger R₁ when the ligand is complexed to the ion than when it is not complexed. Such complexes are of utility as probes in ¹⁹F magnetic resonance applications such as MRI and MRS.

Viewed from one aspect therefore the invention provides a paramagnetic lanthanide (III) complex comprising a lanthanide (III) ion and a polydentate ligand wherein the polydentate ligand comprises one or more fluorine atoms in which the distance of at least one of the fluorine atoms to the lanthanide ion is less than 7 Å, said polydentate ligand not being a DPTA bisamide of p-CF₃-aniline.

Viewed from a further aspect the invention provides the use of a complex according to the first aspect of this invention in ¹⁹F magnetic resonance applications.

Further aspects and embodiments of the invention will be evident from the discussion below.

### BRIEF DESCRIPTION OF THE FIGURES

FIG. 1 shows the pH-dependent variation in the ¹⁹F NMR spectrum of a Europium complex of this invention (298K, 376 MHz, H₂O).

FIG. 2 shows the variation of the ratio of Eu emission band intensities of the same Europium complex depicted in FIG. 1 (λ_{exc} 255 nm, 298K, H₂O, 0.1 M NaCl) versus pH; the inset shows the first derivative plot consistent with an apparent protonation constant of 5.5.

FIG. 3 shows the deprotonation of the amide proton of the holmium complex of ligand L⁴.

FIG. 4 shows the variation of ¹⁹F chemical shift for [Ho.L⁴) with pH at 295 K. Aqueous solution contained 0.1 M NaCl. Solid lines: least-squares fits to the fast-exchange acid-base equilibrium equation for the chemical shift.

FIG. 5 shows the N-coordinated and diaqua species of the holmium (III) complex of ligand L^{2′}.

FIG. 6 shows the variation of the ratio (smaller/greater) of ¹⁹F signal integrals of [Ho.L^{2′}H] and [Ho.L^{2′}]⁻ with pH at 295 K. Aqueous solution contained 0.1 M NaCl. Solid lines: least-squares fits to the slow-exchange acid-base equilibrium equation for the NMR line intensity.

FIG. 7 shows the variation of the observed longitudinal relaxation rate with w_{F} for [Ln.L^{3a}] (Ln=Tb, Dy, Ho and Tm). Solid lines: global least-squares fits to the relaxation theory equations).

### DETAILED DESCRIPTION OF THE INVENTION

The present invention relates to the location of a ¹⁹F nucleus or ¹⁹F nuclei within 7 Å of a paramagnetic lanthanide ion in a complex, for example within 6 Å, or 5.5 Å, or 5 Å from the lanthanide ion. Typically the ¹⁹F nucleus or nuclei will be from 4-7 Å from the lanthanide ion. In some embodiments of the invention all the F atoms will be within one of these distances or ranges of distances, from the lanthanide ion.

For the avoidance of doubt, the invention embraces paramagnetic lanthanide (III) complexes in which at least one of the fluorine atoms is less than 7 Å from the lanthanide ion in a confirmation that the complex may adopt. Thus, for example, we have found that the polydentate ligand may further comprise a linking moiety, such as an amide or sulfonamide moiety, which affects the chemical shift properties of the fluorine atom in a pH-dependent manner. Thus, for example, where the hydrogen atom of the amide or sulfonamide is absent, for example at high pH, the nitrogen atom of the resultant deprotonated amide or sulfonamide may chelate to the lanthanide (III) ion bringing the fluorine atom of the polydentate ligand to within 7 Å or less of the lanthanide (III) ion when this is not the case where the sulfonamide or amide nitrogen atom is protonated.

As such, the invention may provide pH-sensitive paramagnetic complexes that can serve as ¹⁹F chemical shift-based pH probes, where the chemical shift of the ¹⁹F atom within the polydentate ligand chemical shift is a function of pH. Such pH-sensitive complexes are discussed below.

Advantageously, the pseudocontact shift (PCS) (See I. Bertini, C. Luchinat and G. Parigi, Progr. NMR Spec., 2002, 40, 249), induced by the Ln³⁺ ion further amplifies the ¹⁹F chemical shift sensitivity to minor structural changes. Because the PCS and the relaxation properties of spin-1/2 nuclei in paramagnetic environments are well understood (see I. Bertini, F. Capozzi, C. Luchinat, G. Nicastro and Z. C. Xia, J. Phys. Chem., 1993, 97, 6351); (I. Bertini, C. Luchinat and G. Parigi, Progr. NMR Spec., 2002, 40, 249), extra information on distances and geometries may be extracted from the NMR data.

The complexes of this invention comprise a lanthanide (III) ion and a fluorine-containing polydentate ligand in which the polydentate ligand is not a DPTA bisamide of p-CF₃-aniline (DPTA is diethylene triamine pentaacetic acid). In some embodiments the polydentate ligand is not a DPTA bisamide. In other embodiments the polydentate ligand is not based upon DPTA.

The Ln (III) ion may be any of Ce, Pr, Nd, Sm, Eu, Gd, Tb, Dy, Ho, Er, Tm and Yb) e.g. Ce, Pr; Nd, Sm, Eu, Tb, Dy, Ho, Er, Tm and Yb. The Ln (III) ion may be permuted in any given complex. Some of these Ln (III) ions (e.g. Dy, Tm, Ho, Er and Tb, e.g. Dy, Tm and Tb) are strongly relaxing with dipolar and Curie relaxation rates following an r⁻⁶ dependence, where r is the distance between the Ln ion and the F atom; others (e.g. Eu, Yb) may lead to large dipolar shifts, but do not affect relaxation to such an extent, the paramagnetic dipolar shifts following an r⁻³ dependence. The relative effectiveness of the different lanthanide (III) ions at enhancing nuclear relaxation rates or causing dipolar shifts are well known in the literature (see, e.g. I. Bertini and C. Luchinat, Coord. Chem. Rev., 1996, 150, 1; B. Bleaney, J. Magn. Reson., 1972, 8, 91; R. S. Dickins et al., Chem. Rev., 2002, 102, 1977).

The lanthanide ion is generally selected according to the size of the applied magnetic field. Relaxation rate enhancements are most likely to occur with those ions with large magnetic susceptibilities such as Tb, Dy, Ho, Er and Tm. Whilst complexes of Gd(III) may be used in ¹⁹F MR experiments, other lanthanide (III) ions are generally more convenient, because the large increase in R₂ when Gd(III) is used can sometimes reduce detection sensitivity more than is desirable.

Furthermore, the Gd(III) ion is magnetically isotropic, so that the resonance frequency of the proximate fluorine nucleus is not shifted compared to a diamagnetic analogue, such as a complex of Y(III) or La(III). In contrast, the dipolar or pseudocontact shift associated with complexes of Tb, Dy, Ho, Er and Tm, leads to a significant change in the chemical shift of the fluorine nucleus. This feature is particularly pertinent when devising responsive lanthanide probes, in which the fluorine nucleus serves to report local changes in the magnetic environment associated with a perturbation of a given variable, such as pH. For example, the proximate lanthanide(III) ion will amplify any chemical shift non-equivalence in the acid and conjugate base forms of the probe complex. Chemical shift probes of pH are attractive as they lack any dependence on probe concentration. Typically for in vivo work, the pKₐ of the probe complex is within 0.5 pH units of the desired pH range. The pH range in vivo is often between 7.6 and 6.8, but in certain tissues and organs more acidic local environments will occur, e.g. in the kidney or in the GI tract.

By polydentate is meant that the ligand has 6, 7, 8 or 9 coordination sites, or donor atoms, with which to coordinate, or cooperatively bind, to the lanthanide ion. The polydentate ligand may be acyclic or cyclic. Typically the polydentate ligand, however, will be a macrocyclic ligand comprising a polydentate macrocycle and the subsequent invention focuses upon polydentate macrocyclic ligands. The macrocycle itself comprises a closed loop typically made up of 9 to 15 atoms in the backbone of the loop.

In one embodiment, the macrocycle is based upon a 1,4,7,10-tetraazacyclododecane macrocycle, in which there are 12 atoms in the backbone of which the four nitrogen atoms can each serve as a coordination site for the lanthanide ion.

In other embodiments one or more, and preferably three, nitrogen atoms of the macrocycle, for example 1, 4,7,10-tetraazacyclododecane may be substituted with a moiety of the formula

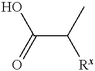

wherein R^{x} is selected from the group comprising H, straight-chain or branched C₁₋₆ alkyl or carboxy (straight-chain or branched) C₁₋₆ alkyl. Preferably R^{x} is selected from the group comprising H, CH₃, CH₂CO₂⁻, CH₂CH₂CO₂⁻ or CH₂CH₂CH₂CO₂⁻.

In one embodiment of the invention the macrocycle comprises a 1,4,7,10-tetraazacyclododecane ligand having a pendant carboxymethyl group attached to three of the four nitrogen atoms.

The macrocyclic ligand also comprises one or more fluorine atoms. These may be present as fluoro substituents—e.g. attached to an alkyl, alkenyl or aryl moiety attached to the macrocycle. Alternatively or additionally the macrocycle may contain a CF₂ or CF₃ moiety. Examples of fluorinated substituents attached to the macrocycles are shown hereinafter; these include mono- and disubstituted phenyl (or other aryl, or heteroaryl) groups such as 4-trifluoromethylphenyl or (2,3)-, (2,4)-, (2,5)-, (2,6)-, (3,4)-, or (3,5)-bis(trifluoromethyl)phenyl.

Examples of the complexes of the invention include those of in which the macrocyclic ligand is of any one of the following formulae:

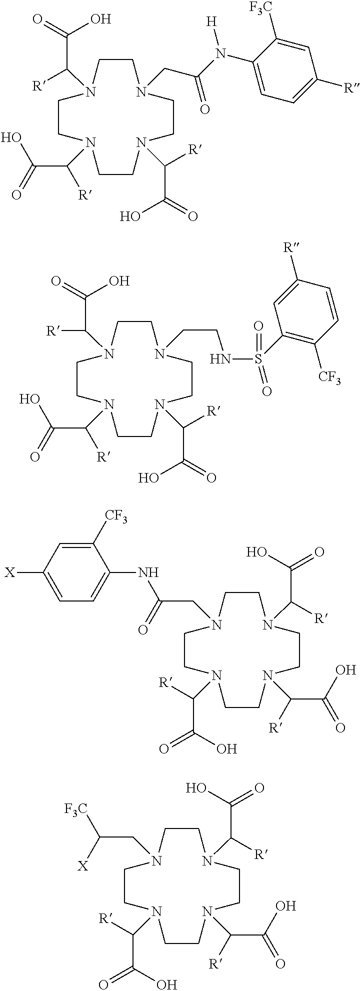

(wherein: each R′ is independently selected from the group comprising H, straight-chain or branched C₁₋₆ alkyl or carboxy (straight-chain or branched) C₁₋₆ alkyl, preferably H, CH₃, CH₂CO₂⁻, CH₂CH₂CO₂⁻ or CH₂CH₂CH₂CO₂⁻; R″ is either selected from the H, CF₃, NO₂, CN, CF₃SO₂, NH₂, NHR^{y} or NR^{y}₂ (wherein R^{y} is selected from the group comprising H, straight-chain or branched C₁₋₆ alkyl or carboxy (straight-chain or branched) C₁₋₆ alkyl; or selected from the group comprising H or CF₃; X is OH, OCOR″′ or O-monosaccharide (e.g. O-β-galactose or O-glucose), NHCOR″′, NHCO-peptide, CO₂R″′, CONHR″′ or CONH-peptide; and R′″ is alkyl or aralkyl).

Peptide refers to a peptidic moiety, which may be either naturally or synthetically derived.

Preferably each R′ is the same.

Further examples of the complexes of the invention are those wherein the macrocyclic ligand is of any one of the following formulae:

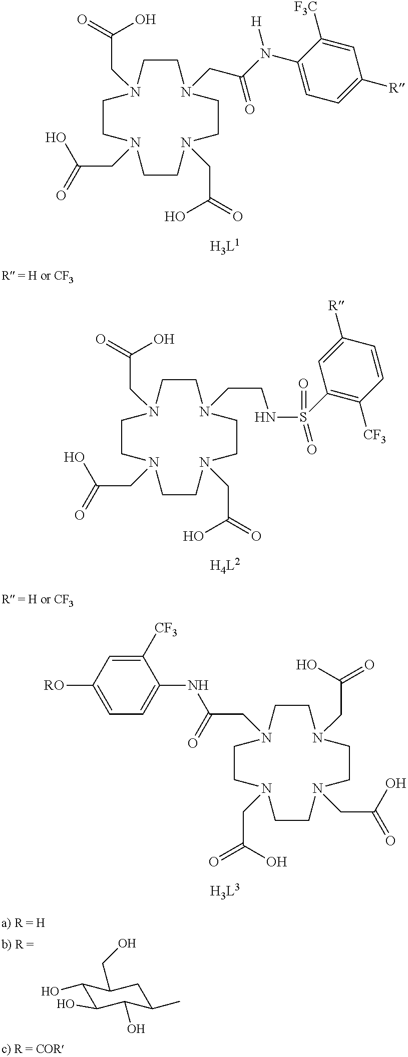

wherein R′ is an optionally substituted alkyl or aralkyl.

The macrocycles immediately hereinbefore described with reference to examples of the complexes of the invention in themselves represent a further aspect of the invention.

The following three sets of fluorinated macrocyclic lanthanide (III) complexes [LnL¹]-[LnL³] have been synthesised and their ¹⁹F NMR spectral properties assessed.

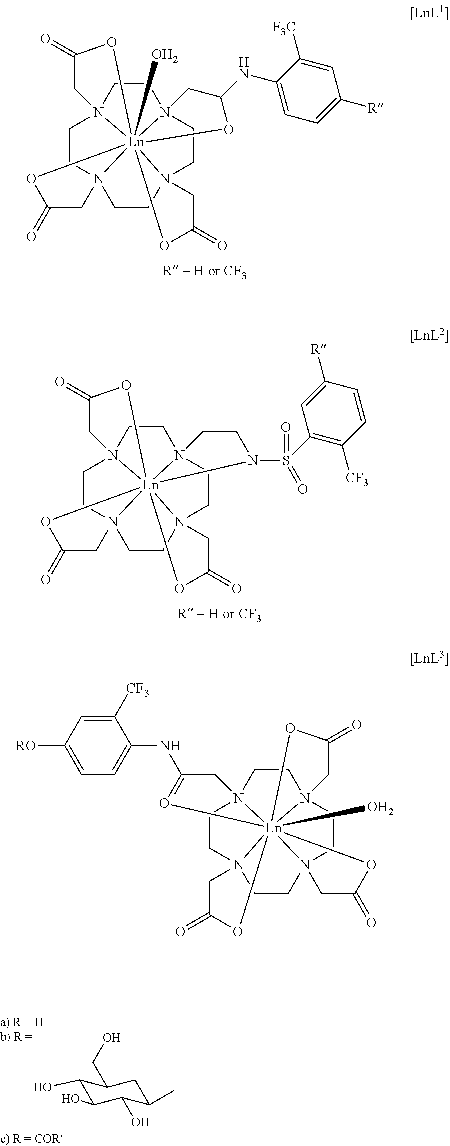

wherein R′ is an optionally substituted alkyl or aralkyl.

Ligands L¹ (R″═CF₃) and L³ form charge neutral mono-aqua complexes with Ln(III) ions (P Caravan et al., infra and R. S. Dickins et al., infra); ligand L² (R″═CF₃) incorporates a sulfonamide moiety that is able to bind reversibly to the Ln ion via the N atom, in a process that is a sensitive function of pH. The exchange between N-bound and the unbound N-protonated ligation modes in water is accompanied by a change in hydration of the Ln ion, and has previously been examined for certain Eu and Gd complexes, varying the protonation constant by changing the electron demand of the sulfonamide substituent. This allows one to define the effective protonation constant of the complex and hence the pH range over which the probe will respond (M. P. Lowe, E. Gianolio, O. Reany, D. Parker, S. Aime and M. Botta, J. Am. Chem. Soc., 2001, 123, 7601; M. P. Lowe and D. Parker, Chem. Commun. 2000, 707).

pH probes are often referred in the art to belong to the “fast-exchange” or “slow-exchange” classes.

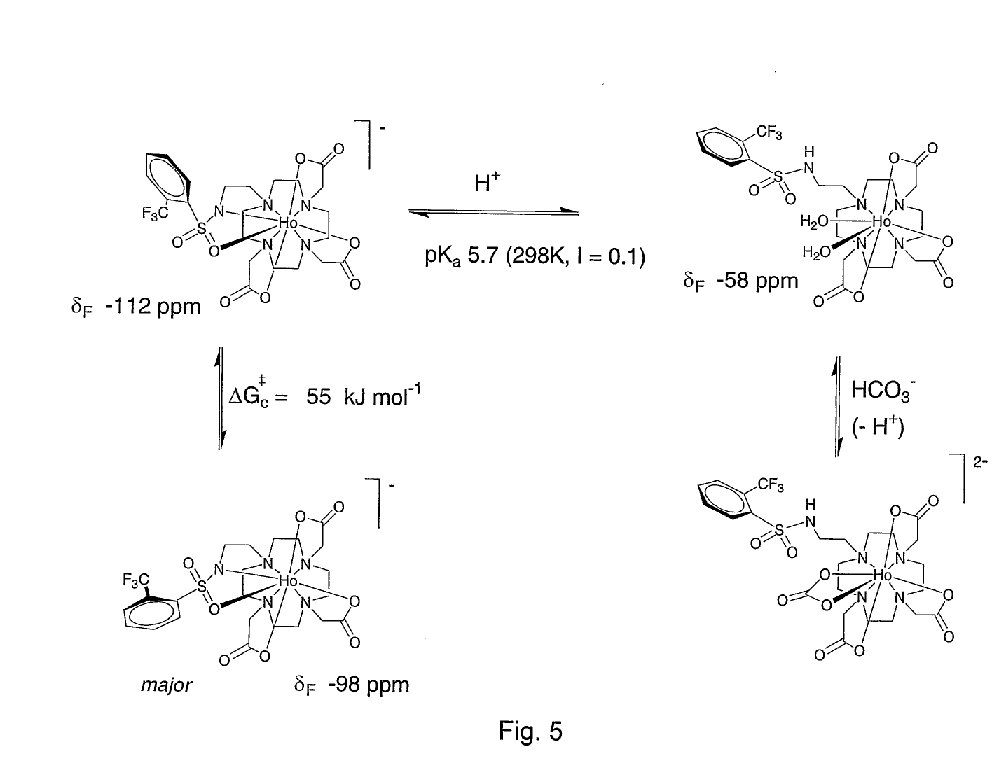

Whilst ligands such as L² described above (wherein R″═CF₃) are able to bind reversibly to the lanthanide ion via the N-atom in a pH dependent process, the resultant complex may be regarded as a “slow-exchange” class of pH probe, in which chemical shifts are pH-independent, and it is relative signal intensities that are altered. Examples of such “slow-exchange” pH probes have been described by P. K. Senanayake, A. M. Kenwright, D. Parker and S. K. van der Hoorn, Chem. Commun., 2007, 2923; M. P. Lowe and D. Parker, Chem. Commun., 2000, 707; and M. P. Lowe, D. Parker, O. Reany, S. Aime, M. Botta, G. Castellano, E. Gianolio and R. Pagliarin, J. Am. Chem. Soc., 2001, 123, 7601). The rationale behind referring to such complexes as “slow-exchange” pH probes may be understood with reference to FIG. 5 in which it may be seen that the basic form of ligand H₄L^{2′}

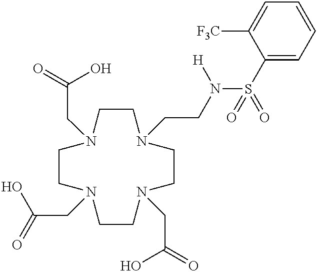

(i.e. having the structure of LnL² above wherein R″═H as opposed to CF₃) and shown in FIG. 5 as complexed with holmium) may exist as a mixture of two diastereoisomers, whose chemical shifts are pH independent but which exist in pH-dependent interrelationship with complexes in which the sulfonamide nitrogen atom is protonated.

An alternative class of pH-sensitive complexes provided by the present invention may be regarded as “fast-exchange” pH probes, in which the chemical shift changes continuously as a function of pH. Examples of such complexes are those where the polydentate ligand comprises a moiety that affects, in a pH-dependent manner, the disposition of the one or more fluorine atom relative to the lanthanide (III) ion in the complex whereby to affect the chemical shift of the fluorine atom by ¹⁹F NMR.

Examples of such complexes have formulae related to complex [LnL¹] wherein the phenyl ring is substituted at the para position relative to the amide group with a substituent capable of increasing the acidity of the amide hydrogen atom. An example of such a substituent is the nitro group, which renders the amido hydrogen atom susceptible to deprotonation under basic conditions. Other examples of substituents capable of increasing the acidity of the amide hydrogen atom (of comparable electron-withdrawing ability) include CN and SO₂CF₃ groups.

Experimental measurements and theoretical analysis of magnetic properties, structural dynamics and acid-base equilibria for several lanthanide (III) complexes with tetraazacyclododecane derivatives as ¹⁹F NMR chemical shift pH probes are described below; together with their relaxation behaviour as a function of magnetic field, temperature and the nature of the lanthanide ion. We report titration curves and pH dependence of the ¹⁹F chemical shift in water, mouse urine and murine blood plasma. Typical pKₐ values vary between about 6 and 8, with pKₐs from 6.9 to 7.7 exemplified below, with 18 to 40 ppm chemical shift differences between the acidic and basic forms for Ho(III) complexes possessing T₁ values of 10 to 30 ms (4.7 to 9.4 Tesla, 295 K).

Thus the para substituent of the aromatic ring of these ligands H₃L¹ and H₃L³, for example regulates the acidity of the amide hydrogen and therefore the pH sensitivity of the complex. Consistent with the low acidity (pKₐ>9.5) of the amide proton of an analogue of the H₃L¹ ligand in which R″ is not H or CF₃ but an amino or hydroxyl group (ligand H₃L⁵ and H₃L^{3a} respectively), no pH dependence of the ¹⁹F chemical shift of [Ln.L⁵] and [Ln.L^{3a}] was observed over the pH range of 3.5 to 9.0. The corresponding para nitro-substituted ligand (L⁴), however, is significantly more acidic.

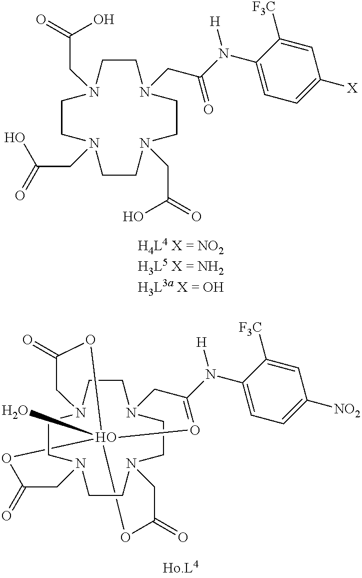

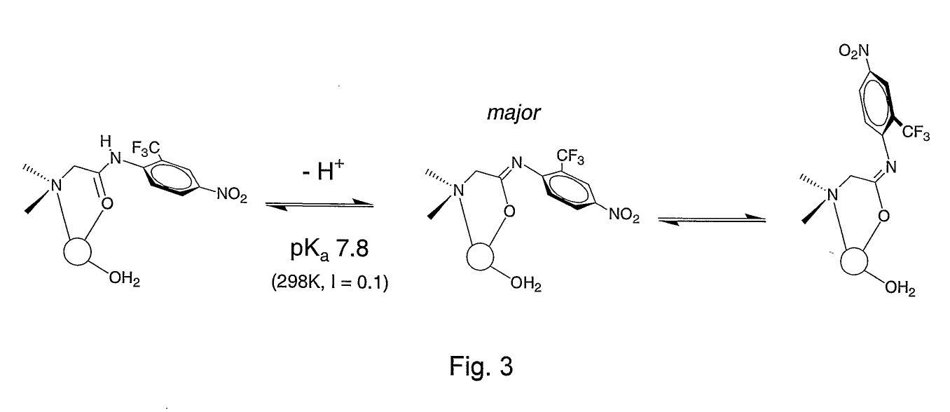

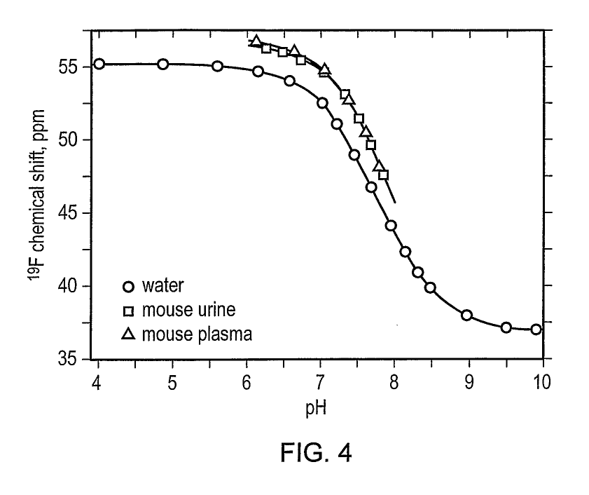

The NH group of [Ln.L⁴] deprotonates in alkaline conditions, causing the CF₃ group of [Ho.L⁴] to shift from −55.1 ppm to −36.8 ppm under basic conditions (see FIG. 3). Because the acid-base equilibrium is fast in this system (noting and reporting lanthanide re-coordination involved), only one signal is observed, with the chemical shift corresponding to the weighted average of the chemical shifts of the individual forms. Fitting the acid-base equilibrium curve to the experimental chemical shift data (FIG. 4) resulted in a pKₐ=7.77±0.02, in 0.1 M NaCl solution.

While the longitudinal ¹⁹F relaxation rate of [Ho.L⁴] is nearly pH-independent, we observed an abrupt increase (by a factor of 8) in the linewidth (see Table 3 below), which disappears upon heating, indicating the presence of an intermediate timescale chemical exchange process in the deprotonated complex. Two possible chemical exchange processes were considered: lanthanide re-coordination, from oxygen to nitrogen, or cis-trans isomerization around the >C═N— double bond (see FIG. 3). Based on DFT calculations (see Example 4 below), lanthanide re-coordination can be ruled out, as the DFT energy difference between the two coordination isomers of [La.L⁴] is 62 kJ/mol in favour of the oxygen-bound isomer. However, the calculated energy difference between the cis- and trans-isomers is only 18 kJ/mol, making the cis-trans isomerisation a more likely explanation for the additional ¹⁹F signals observed. Based on the saddle point energy, the forward and backward activation energies for the cis-trans isomerization are 27 kJ/mol and 45 kJ/mol, consistent with the exchange broadening observed in the ¹⁹F NMR spectra. The calculations highlighted the important role of the bulky CF₃ group in favouring rotamers that place the CF₃ group away from the amide oxygen.

In aqueous solution, two signals with pH-dependent intensities and 40 ppm chemical shift difference were observed for [Ho.L^{2′}], corresponding to N-coordinated and diaqua species (see FIG. 4). Fitting the acid-base equilibrium equation to the titration data (see FIG. 5) yielded pKₐ values of 5.71±0.02 (water), 6.88±0.02 (murine urine) and 6.92±0.02 (murine plasma). The higher pKₐ values in biofluids are the result of additional stabilization of the acidic form by carbonate coordination (see FIG. 4) (S. Aime, A. Barge, M. Botta, J. A. K. Howard, R. Kataky, M. P. Lowe, J. M. Moloney, D. Parker and A. S. de Sousa, Chem. Commun., 1999, 1047); (Y. Bretonniere, M. J. Cann, D. Parker and R. J. Slater, Org. Biomol. Chem., 2004, 2, 1624). The basic form exists as a mixture of two diastereomers, defined by the R or S configuration at the sulphur atom (Scheme 3). The experimental ratio is 6:1 (298 K, pH=8), resonating at −98 and −112 ppm respectively. A slow re-coordination process interconverts these two species: EXSY spectroscopy and a variable temperature ¹⁹F NMR study revealed an exchange process (T_{c} 323K at 376 MHz), that probably occurs via a cooperative S═O-Ln bond cleavage/reformation. DFT calculations confirmed this hypothesis—the computed energy difference between the two coordination isomers (via either oxygen at the stereogenic S centre) is 8.9 kJ/mol for [La.L^{2′}]. A first-order saddle point was found between the two structures, corresponding to forward and backward activation energies of 85 and 76 kJ/mol (see Example 4 below for DFT calculation details).

Thus the invention advantageously provides two new classes of pH-sensitive ¹⁹F-labelled paramagnetic complexes. With holmium (III), a 40 ppm ([Ho.L^{2′}]) and 18.3 ppm ([Ho.L⁴]) change in chemical shift was observed between the acidic and the basic form. [Ho.L⁴] belongs to the ‘fast-exchange’ type and changes its ¹⁹F chemical shift with pH, whereas [Ho.L^{2′}] is a ‘slow-exchange’ ratiometric probe, responding to pH changes by variation of relative signal intensities.

The complexes of the present invention are of use in media containing high concentrations of salts and/or high concentrations of protein, as may be found in the analysis of samples of environmental, clinical or biological interest, e.g. urine or blood (e.g. blood plasma) samples.

The macrocycles may also further comprise a functional group susceptible to modification, for example by an enzyme. Thus, for example, the macrocycles may contain an amide, or a carboxylic acid, glycoside or phosphate ester subject to hydrolysis by an enzyme.

The complexes of this invention allow the rate of relaxation of the ¹⁹F nucleus to be enhanced. The enhancement varies depending upon the specific complex but, typically is by at least a factor of 5, often by a factor of 10 and in some cases by a factor of 100 or more. Such enhancements allow the acquisition of more scans and hence increased spectral signal intensity in a given time period for a given concentration of complex. Thus, the sensitivity of ¹⁹F MRS or MRI studies is considerably enhanced, allowing the use of less concentrated solutions containing these fluorinated lanthanide (III) complexes.

With the fluorine nucleus close in space to the paramagnetic lanthanide (III) ion, the resonance frequency of the ¹⁹F nucleus may be shifted by dipolar coupling, by a large value compared to a diamagnetic analogue, e.g. of the order of up to 100 ppm for Dy and Tb(III) complexes. This effect serves to enhance any ¹⁹F chemical shift non-equivalence in the ligand fluorine resonances, for cases where the ligand structure is slightly modified, for example following the action of an enzyme on certain functional groups the ligand structure, e.g. enzyme-catalysed hydrolysis of an integral carboxylic acid ester, amide, or a glycoside or phosphate ester.

In order to use a fluorinated paramagnetic probe to follow enzyme activity (and thereby signal gene expression), it is sufficient to be able to distinguish by ¹⁹F NMR between the substrate and the product of enzyme action. Thus, ester, amide or glycoside bonds may be incorporated into the fluorinated paramagnetic probe structure to serve as the substrate for the given enzyme. As an example of this the ¹⁹F chemical shift non-equivalence of the CF₃ reporter group in [LnL^{3c] above (wherein R′=Me) vs the product of cleavage of the ester bond, [LnL}^{3a}] is sufficient (Δδ_{F}=5.3 ppm for the Tm example in Table 1) to allow this complex to report on the activity of esterase enzymes that catalyse hydrolysis of the ester bond in the [LnL^{3b}] complex. The use of related systems to monitor glycosidase, protease, phosphatase or lipase activity may be envisaged in a similar manner, by including, glycoside, amide, phosphate ester or carboxyalkyl groups into the structure of the fluorinated probe complex, monitoring enzyme activity or gene expression by ¹⁹F MRS or ¹⁹F chemical shift imaging. The monitoring may be in vivo or ex vivo for example in vitro.

The following non-limiting examples are intended to illustrate various aspects and embodiments of the invention.

### Fluorine-Lanthanide Distances

As is known to those skilled in the art, the fluorine-lanthanide distances may be obtained from the field dependence of the R₁ₚ values (I. Bertini, F. Capozzi, C. Luchinat, G. Nicastro and Z. C. Xia, J. Phys. Chem., 1993, 97, 6351); I. Bertini, C. Luchinat and G. Parigi, Progr. NMR Spec., 2002, 40, 249); which was performed for the L^{3a} ligand complexes. The dominant relaxation mechanisms for paramagnetic systems in question are electron-nucleus dipole-dipole (DD) and Curie processes (CSR). The total longitudinal relaxation rate (weak perturbation limit, isotropic tumbling approximation, DD-CSR cross-correlation neglected) is (see I. Bertini et al. infra).

$${R}_{1} = \frac{2}{15} {( \frac{{μ}_{0}}{4 π} )}^{2} \frac{{γ}_{F}^{2} {μ}_{eff}^{2}}{{r}^{6}} [ \frac{7 {τ}_{r + e}}{1 + {ω}_{e}^{2} {τ}_{r + e}^{2}} + \frac{3 {τ}_{r + e}}{1 + {ω}_{F}^{2} {τ}_{r + e}^{2}} ] + \frac{2}{5} {( \frac{{μ}_{0}}{4 π} )}^{2} \frac{{ω}_{F}^{2} {μ}_{eff}^{2}}{{( 3 kT )}^{2} {r}^{6}} \frac{3 {τ}_{r}}{1 + {ω}_{F}^{2} {τ}_{r}^{2}}$$

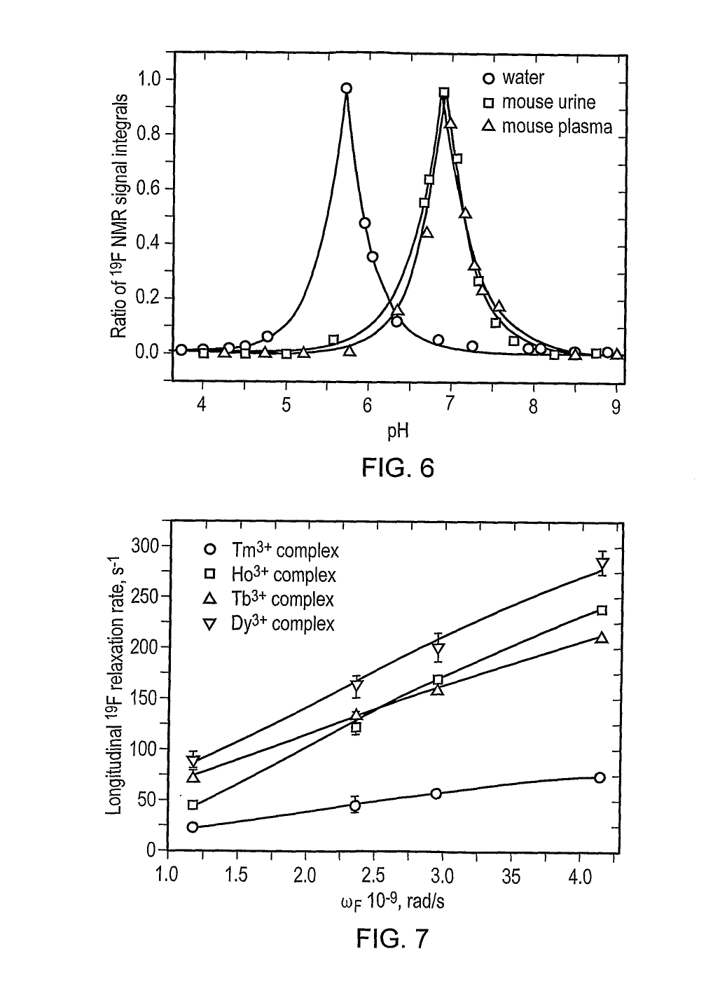

Because the geometry does not change significantly between different lanthanides (ionic radius variation is minor—Tb³⁺ 1.09 Å, Dy³⁺ 1.08, Ho³⁺ 1.07 and Tm³⁺ 1.05 Å in 9 coordinate systems (R. D. Shannon, Acta Cryst. A, 1976, 32, 751) the Ln-F distance and the rotational correlation time can be set to be global between the Tb, Dy, Ho and Tm data sets (full tables are given in Example 4 below). The values of μ_{eff} and τᵣ₊ₑ are kept ‘local’ for every lanthanide dataset. While each individual fit is ambiguous, the global fit (FIG. 6) has a single, well-defined, weighted least-squares minimum. The resulting values are r_{Ln-F}=6.9±0.8 Å, τᵣ=280±12 ps (alternative fitting using fixed values of μ_{eff}: 7.6 for Tm³⁺, 10.6 for Ho³⁺, 9.7 for Tb³⁺ and 10.6 for Dy³⁺ results in r_{Ln-F}=6.24±0.01 Å, τᵣ=286±12 ps), compared with the Stokes-Einstein rotational correlation time estimated from DFT volume data τᵣ=243 ps (rₘₒₗ=6.18 Å) and the DFT Ln-F distance of <r>=6.95 Å.

### Example 1

### Fast Relaxation and Enhanced Signal Intensity

¹⁹F NMR chemical shifts and longitudinal relaxation rates for various lanthanide complexes of L¹, L² and L³ were measured at 188 MHz (4.7 T, 298K).

The diamagnetic yttrium (III) complex of ligand L¹ serves as a useful reference point. Both of the CF₃ groups in this reference example had slow relaxation rates of the order of 0.9 s⁻¹.

Both the dipolar shifts and the relaxation rate enhancements were largest for the ortho-CF₃ groups that are closest in space to the paramagnetic lanthanide (III) ion: in [TbL¹], the rate of relaxation was 70 s⁻¹ (Table 1) and for [DyL²]⁻ the CF₃ resonance shifted 100 ppm to lower frequency (of the ligand) and the measured R₁ value was 93 s⁻¹.

TABLE 1
Limiting ¹⁹F chemical shift (ppm) and longitudinal
relaxation rate (s⁻¹) data (188 MHz, 298K, pH 5.5) for
selected lanthanide complexes of L¹, L² and L³

| | δ_{F•}^{c} | δ_{F∘}^{c} | | |
|---|---|---|---|---|
| | ortho CF₃ | other CF₃ group | | |
| Complex | group | if present | R₁^{•} | R₁^{∘} |
| [TbL¹] | −50.9 | −74.7 | 90 | 11 |
| [YbL¹] | −65.4 | −59.6 | 4.6 | 1.7 |
| [TmL¹] | −79.6 | −55 | 16 | 5.6 |
| [YL¹] | −61.3 | −63.1 | 0.9 | 0.9 |
| [DyL¹] | −65.0 | −81.2 | 77 | 12 |
| [EuL²]^{-a} | −53 (−59) | −65 (−64) | 2.5 (2.2) | 1.5 (1.2) |
| [TbL²]^{-a,b} | −159 (−59) | −45 (−64) | 71 | 23 |
| [DyL²]^{-a,b} | −158 (−59) | −39 (−64) | 93 | 27 |
| [TmL^{3a}] | 77.0 | — | 24 | — |
| [TmL^{3b}] | 71.7 | — | 17 | — |

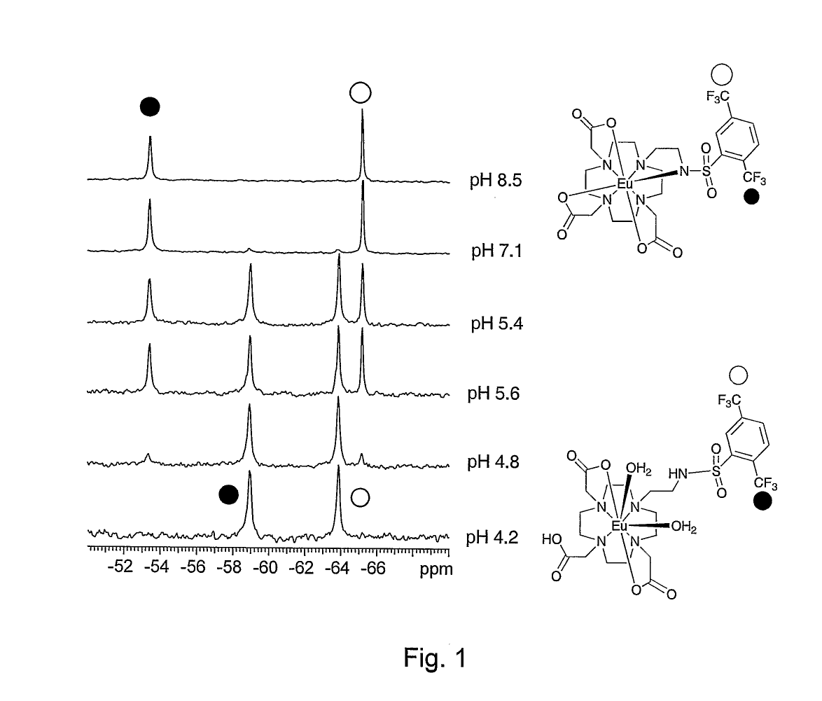

^{a}the lower rate enhancements for Eu compared to other ions in this Table, reflects its lesser intrinsic ability to enhance the rate of nuclear relaxation (Bertin/Luchinat et al.; Dickins et al.); values in parentheses refer to the N-protonated complex (pH 5.5, see FIG. 1);
^{b}for [DyHL²] and [TbHL²] , the resonances of the two CF₃ groups were exchange broadened and were not distinguished at pH 5.5 (188 MHz), and mean rates of 23 s⁻¹ and 27 s⁻¹ were measured respectively;
^{c}shifts were internally referred to NaCF₃CO₂ (δ_{F}-76.1 ppm) which typically had a relaxation rate of 0.4 s⁻¹ under the stated conditions.

The distinct advantages of introducing the Ln ion are to amplify any chemical shift non-equivalence in the reporter resonances and to enhance the rate of relaxation allowing acquisition of more scans and hence increased signal intensity in a given time period. A comparative analysis examined the ortho-CF₃ resonance in ¹⁹F NMR spectra of the diamagnetic Y complex, [YL¹] (R₁ 0.93 s⁻¹, {linewidth, ω_{1/2}=2 Hz}) and the terbium (R₁ 90 s⁻¹ {.ω_{1/2} 90 Hz} and Dy (R₁ 77 s⁻¹ {.ω_{1/2} 35 Hz}) analogues. Using a relaxation delay of 3 times T₁, ¹⁹F spectra were acquired over a common time interval of 30 minutes (2 mM complex, 188 MHz (4.7 T), 298K, typical S/N ratio per mM was 100:1). The relative signal intensity ratios per mM of complex (peak height times peak width at half height; processing used a line-broadening function equal to half of the observed linewidth, for the given complex resonance) for observation of the major ortho-CF₃ resonance in [TmL¹], [DyL¹] and [TbL¹] versus the diamagnetic analogue, [YL¹], were 52:1 22:1 and 41:1 respectively. This is consistent with the ability to increase the number of scans acquired for the faster relaxing CF₃ resonance in the paramagnetic complexes.

### Example 2

### A ¹⁹F NMR Chemical Shift Probe

The NMR spectral behaviour is slightly more complex for the [LnL²]⁻/[LnLH] system, as chemical exchange between these species leads to additional line broadening. In this example, the chemical exchange process was in the slow-exchange regime on the NMR timescale; taking [EuL²]⁻/[EuL²H] as an example, separate shifted ¹⁹F resonances were observed for each complex as shown in FIG. 1.

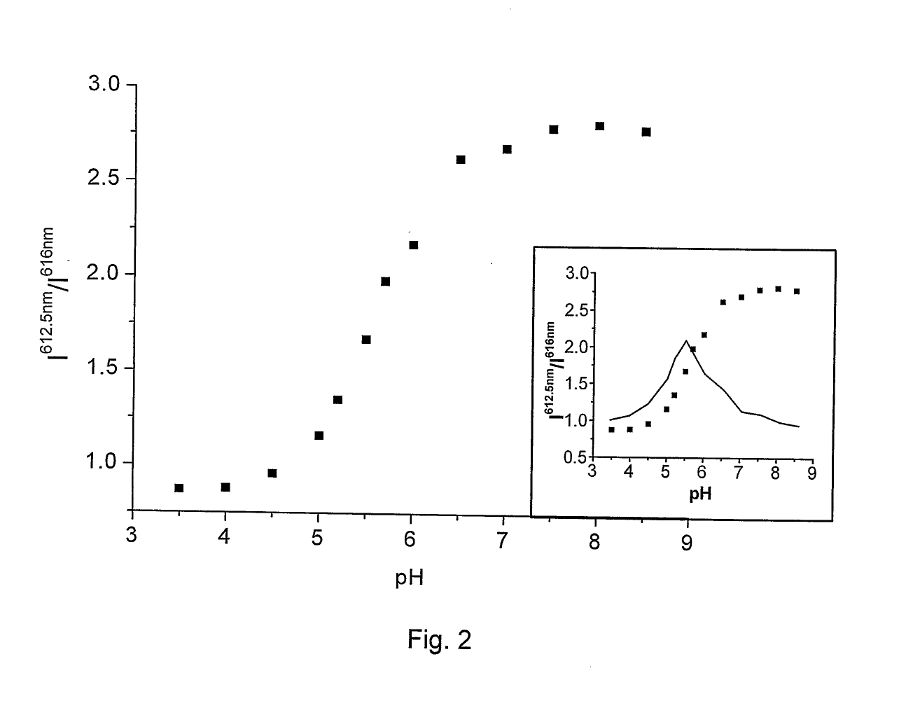

Their co-observation allowed the complex to function as a chemical shift pH probe. Thus, the ratio of the integrated intensities of the pairs of resonances at −59/−53 and −65/−64 ppm reflects the relative concentration of the conjugate base and its protonated form, allowing determination of the complex protonation constant, pK_{MLH}=5.5 (±0.05). Independent validation of this value comes from an analysis of the variation of the Eu luminescence emission intensity ratio (λ_{exc} 397 nm; observe the ratio of the 612/616 nm or 680/590 nm bands) with pH (see FIG. 2 which shows variation of the ratio of Eu emission band intensities (λ_{exc} 255 nm, 298K, H₂O, 0.1 M NaCl) versus pH; the inset shows the first derivative plot consistent with an apparent protonation constant of 5.5).

### Example 3

### Measurement of ¹⁹F Chemical Shifts and Relaxation Parameters^{a} at 295 K for Lanthanide (III) Complexes with Ligands L^{3a} to L⁵ and L^{2′}

These are shown in Table 2 below at 9.4 Tesla with selected lanthanide ions.

TABLE 2

| Complex | [HoL⁴H] | [HoL⁴]⁻ | [HoL^{2′}H] | [HoL^{2′}]⁻ | [TbL⁵] |
|---|---|---|---|---|---|
| δ_{F}/ppm | −55.1 | −36.8 | −58.0 | −98.1 | −53.0 |
| R₁ @ 9.4 T/s⁻¹ | 92 ± 9 | 116 ± 21 | 112 ± 6 | 128 ± 5 | 125 ± 6 |
| R₂^{c} @ 9.4 T/s⁻¹ | 179 | 1490^{d} | 210 | 989^{e} | 272 |

| Complex | [TbL^{3a}]^{b} | [DyL^{3a}]^{b} | [HoL^{3a}]^{b} | [TmL^{3a}]^{b} |
|---|---|---|---|---|
| δ_{F}/ppm | −52.5, | −66.7, | −57.9, | −78.1, |
| | −40.6 | −43.8 | −49.0 | −89.2 |
| R₁ @ 9.4 T/s⁻¹ | 133 ± 7 | 162 ± 11 | 124 ± 10 | 47 ± 7 |
| R₂^{c} @ 9.4 T/s⁻¹ | 206 | 355 | 192 | 89 |

^{a}Diamagnetic complexes (with La³⁺ or Y³⁺) with the same ligands under the same conditions have R₁ ~1 s⁻¹ and R₂ ~3 s⁻¹.
^{b}The share of minor species (at the second chemical shift quoted): 12% (Tb), 12% (Ho), 9% (Tm) and 20% (Dy).
^{c}R₂ values estimated as 2p · (half-width@half-height).
^{d}Intermediate exchange regime between two cis-trans isomers. See infra for further details.
^{e}Intermediate exchange regime between two coordination isomers. See infra for further details.

Further relaxation analysis data are the field range 4.7 T to 16.5 T was obtained as follows:

¹⁹F T₁ times were measured in dilute D₂O solutions at 295 K using the inversion-recovery technique, without proton decoupling, on Varian spectrometers operating at magnetic inductions corresponding to proton frequencies of 200, 400, 500 and 700 MHz (Mercury-200, Mercury-400, Inova-500, VNMRS-700). The resulting free induction decays were subjected to backward linear prediction, optimal exponential weighting, zero filling, Fourier transform, phasing and baseline correction (by polynomial fitting to signal-free spectrum areas). The signals were integrated by Lorentzian line fitting. Inversion-recovery type function was fitted to the resulting data using Levenberg-Marquardt minimization of the non-linear least squares error functional.

TABLE 3
Longitudinal relaxation rates for four [Ln•L^{3a}] complexes.

| ¹⁹F NMR | | | | |
|---|---|---|---|---|
| frequency, | Longitudinal relaxation rate, s⁻¹ | | | |
| MHz | [Ho•L^{3a}] | [Tb•L^{3a}] | [Tm•L^{3a}] | [Dy•L^{3a}] |
| 188.16 | 45.5 ± 2.5 | 74.4 ± 0.4 | 22.9 ± 0.5 | 88.9 ± 8.4 |
| 376.31 | 124.4 ± 9.5 | 132.5 ± 7.0 | 46.7 ± 7.0 | 162.4 ± 11.3 |
| 470.25 | 169.0 ± 1.9 | 161.7 ± 0.5 | 57.5 ± 0.7 | 201.3 ± 13.5 |
| 658.41 | 238.7 ± 1.6 | 211.1 ± 0.5 | 74.4 ± 0.5 | 286.3 ± 12.7 |

TABLE 4
Transverse relaxation rates, estimated as 2π• (half-width@half-height),
of a Lorentzian line fit) for four [Ln•L^{3a}] complexes.

| | |
|---|---|
| ¹⁹F NMR | |
| frequency, | Transverse relaxation rate, s⁻¹ |

| MHz | [Ho•L^{3a}] | [Tb•L^{3a}] | [Tm•L^{3a}] | [Dy•L^{3a}] |
|---|---|---|---|---|
| 188.16 | 87 | 124 | 53 | 156 |
| 376.31 | 192 | 206 | 89 | 355 |
| 470.25 | 267 | 271 | 112 | 543 |
| 658.41 | 441 | 407 | 168 | 740 |

### Example 4

### DFT Calculations

The DFT calculations were performed using the Gaussian03 package for La³⁺ and Y³⁺ complexes—the structures of complexes with other lanthanides as well as those with Y³⁺ are known to be nearly identical. Importantly, however, the use of diamagnetic La³⁺ and Y³⁺ for DFT calculations avoids a host of largely unresolved theoretical issues with spin-orbit coupling and zero-field splitting in open-shell lanthanides. Y³⁺ results are nearly identical to the La³⁺ results, therefore only the latter are tabulated below. Gaussian03 logs and checkpoints are available from IK upon request.

Molecular geometries were optimized in vacuo using spin-restricted B3LYP exchange-correlation functional with a compound basis set (cc-pVDZ for CHNOFS, Stuttgart ECP28MWB for Ln and WGBS for Y). Saddle points were located using QST2 and QST3 methods. Hessians were computed and intrinsic reaction coordinates traced in both directions to ensure that the saddle points located are first-order saddles corresponding to the process under consideration.

TABLE 5
DFT B3LYP energies and their complete basis set (CBS) extrapolation
for the two coordination isomers of [La•L⁴]⁻.

| | N-coordinated | O-coordinated |
|---|---|---|
| Basis | isomer energy^{c}, | isomer energy^{c}, |
| set^{a} | Hartree | Hartree |
| cc-pVDZ | −2710.29498 | −2710.31791 |
| cc-pVTZ | −2710.98166 | −2711.00458 |
| cc-pVQZ | −2711.16717 | −2711.19051 |
| CBS limit^{b} | −2711.23584 | −2711.25955 |

^{a}The basis set quoted is for HCNOFS, the basis set for La is Stuttgart RSC ECP28MWB basis set in all cases.
^{b}Dunning-Feller extrapolation.
^{c}Geometries optimized with cc-pVDZ (HCNOFS) and Stuttgart RSC ECP28MWB (La) basis set.

TABLE 6
DFT B3LYP energies and their complete basis set (CBS) extrapolation
for the two constitutional isomers of [La•L⁴]⁻ complex.

| | “Ring-down” | “Ring-up” | Saddle point |
|---|---|---|---|
| Basis | isomer energy^{c}, | isomer energy^{c}, | energy^{c}, |
| set^{a} | Hartree | Hartree | Hartree |
| cc-pVDZ | −2710.31084 | −2710.31791 | −2710.30056 |
| cc-pVTZ | −2710.99738 | −2711.00458 | −2710.98864 |
| cc-pVQZ | −2711.18342 | −2711.19051 | −2711.17477 |
| CBS limit^{b} | −2711.25257 | −2711.25955 | −2711.24379 |

^{a}The basis set quoted is for HCNOFS, the basis set for La is Stuttgart RSC ECP28MWB basis set in all cases.
^{b}Dunning-Feller extrapolation.
^{c}Geometries optimized with cc-pVDZ (HCNOFS) and Stuttgart RSC ECP28MWB (La) basis set, saddle point located with QST2 method, Hessians and IRCs checked.

TABLE 7
DFT B3LYP energies and their complete basis set (CBS)
extrapolation for the two coordination isomers of
[La•L^{2′}]⁻ (“sulphonamide”) complex.

| | Isomer A | Isomer B | Saddle point |
|---|---|---|---|
| Basis | energy^{c}, | energy^{c}, | energy^{c}, |
| set^{a} | Hartree | Hartree | Hartree |
| cc-pVDZ | −2903.85656 | −2903.86062 | −2903.82499 |
| cc-pVTZ | −2904.53840 | −2904.54175 | −2904.50889 |
| cc-pVQZ | −2904.72079 | −2904.72411 | −2904.69169 |
| CBS limit^{b} | −2904.78740 | −2904.79078 | −2904.75837 |

^{a}The basis set quoted is for HCNOFS, the basis set for La is Stuttgart RSC ECP28MWB basis set in all cases.
^{b}Dunning-Feller extrapolation.
^{c}Geometries optimized with cc-pVDZ (HCNOFS) and Stuttgart RSC ECP28MWB (La) basis set, saddle point located with QST2 method, Hessians and IRCs checked.

### Example 5

### Ligand and Complex Synthesis

The following experimental details serve to illustrate the preparation of lanthanide (III) complexes of ligands L¹ to L⁵ above, with appropriate characterisation:

### 2-Chloro-N-(2,4-bistrifluoromethylbenzene)-ethanamide

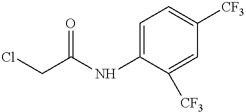

Chloroacetylchloride (1.26 ml, 15.6 mmol) was added dropwise to a stirred solution of 2,4-(bistrifluoromethyl) aniline (1.02 ml, 6.6 mmol) and triethylamine (1.08 ml, 7.8 mmol) in dry diethyl ether (35 ml) at −20° C. The reaction mixture was allowed to warm to room temperature and stirred for 24 hours. The resulting white precipitate was dissolved in water (30 ml) and the organic layer was washed with hydrochloric acid (0.1 moldm⁻³, 30 ml) followed by water (3×20 ml) then dried over K₂CO₃. After filtration the solvent was removed under reduced pressure to yield a white solid. The product was purified by column chromatography over silica gel (1:2 toluene:hexane) giving a white crystalline solid (1.41 g, 69%), m.p. 91-92° C. R_{f} (1:2 toluene:hexane) 0.13; νₘₐₓ (NaCl)/cm⁻¹ 3274 (N—H), 1680 (C═O), 1570, 1500 (N—H), 1260 (C—F), 1100 (C—N); δ_{H} (200 MHz, CDCl₃) 4.25 (2H, s, CH₂Cl), 7.82-7.90 (2H, m, H5 and 6), 8.54 (1H, s, H3), 8.94 (1H, br, s, NH); δ_{C} (50 MHz, CDCl₃) 43.02 (CH₂), 120.09-137.79 (aromatics and CF₃'s), 164.60 (C═O); δ_{F} (188 MHz, CDCl₃) −63.04 (p-CF₃), −61.57 (o-CF₃); m/z (electrospray, ES⁻) 304.1 (100%, [M−H]⁻), 339.6 (4%, [M−H+Cl]⁻). Found: [M−H]⁻, 303.9969. C₁₀H₅ON³⁵Cl₆F₆ requires [M−H]⁻, 303.9964. Found: C, 39.39; H, 2.03; N, 4.40. C₁₀H₆OF₆NCl requires C, 39.30; H, 1.98; N, 4.58%.

### 10-[(2,4-bis-trifluoromethylphenyl)carbamoylmethyl]--1,4,7-tris(tert-butoxycarbonylmethyl) 1,4,7,10-tetraazacyclododecane

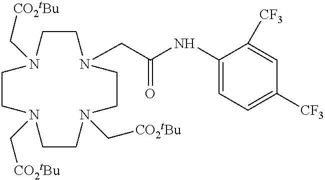

To 1,4,7-tris(tert-butoxycarbonylmethyl)-1,4,7,10-tetraaza cyclododecane (0.421 g, 0.82 mmol) and potassium carbonate (0.125 g, 1.0 mmol) in the presence of 4 Å molecular sieves, under argon, was added a solution of 2-chloro-N-(2,4-bistrifluoromethylphenyl)ethanamide (0.250 g, 0.82 mmol) in dry acetonitrile (30 ml) at room temperature. The mixture was boiled under reflux for 24 hours. The inorganic salts and molecular sieves were removed by filtration and the residue washed with dichloromethane (2×30 ml). The solvents were then removed under reduced pressure and the residue purified by column chromatography over silica gel, eluting with DCM and then up to 4% MeOH/DCM. The solvent was removed under reduced pressure to yield the protected ligand (0.427 g, 54%) as a white semi-crystalline solid, m.p. 186-189° C. R_{f} (2% MeOH, 98% DCM) 0.13; δ_{H} (200 MHz, CDCl₃) 1.44 (27H, s, ^{t}Bu), 2.88-2.91 (12H, m, br, cyclen ring), 3.09 (4H, s, cyclen ring), 3.28 (2H, s, acetate CH₂), 3.37 (4H, s, acetate CH₂'s), 3.75 (2H, s, CH₂C═O), 7.69 (1H, d, J8.6, aromatic H6), 7.77 (1H, d, J8.6, aromatic H5), 7.84 (1H, s, aromatic H3), 10.22 (1H, s, NH); δ_{C} (50 MHz, CDCl₃) 28.05 (6×CH₃), 28.36 (3×CH₃), 47.69 (2×NCH₂), 49.30 (2×NCH₂), 51.45 (2×NCH₂), 53.69 (2×NCH₂), 55.66 (acetate CH₂), 55.85 (acetate CH₂), 56.86 (acetate CH₂), 58.28 (CH₂C═O), 81.81 (C(CH₃)₃), 81.99 (C(CH₃)₃), 82.09 (C(CH₃)₃), 123.91-139.19 (aromatics and CF₃'s), 169.84 (C═O), 170.75 (2×C═O), 172.70 (NC═O); δ_{F} (188 MHz, CDCl₃) −63.2 (p-CF₃), −61.2 (o-CF₃); m/z (electrospray, ES⁺) 784.4 (15%, [M+H]⁺), 806.4 (100%, [M+Na]⁺), 807 (40%). Found [M+H]⁺, 784.4019. C₃₆H₅₆O₇N₅F₆ requires [M+H]⁺, 784.4014.

### 10-[(2,4-bis-trifluoromethylphenyl)carbamoylmethyl]--1,4,7-tris(carboxymethyl) 1,4,7,10-tetraazacyclododecane, L¹

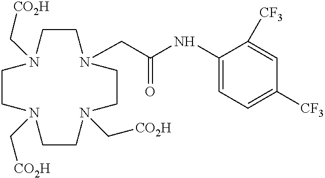

10-[(2,4-bis-trifluoromethylphenyl)carbamoylmethyl]--1,4,7-tris(tert-butoxycarbonylmethyl 1,4,7,10-tetraazacyclododecane (0.200 g, 0.255 mmol) in trifluoroacetic acid (10 ml) and DCM (10 ml) was stirred at room temperature for 12 hours. The solvent was removed under reduced pressure and the resulting solid washed with DCM (3×5 ml) and the solvent removed on the vacuum line. The acid was then washed with DCM (5 ml), the solvent removed by pipette, dried under reduced pressure, taken into water (5 ml) and freeze dried to yield the ligand (0.153 g) as a colourless solid, m.p. 166-168° C. R_{f} (98% DCM, 2% methanol) 0.34; δ_{H} (200 MHz, D₂O) 2.70-3.43 (16H, m, br, cyclen ring), 3.47-3.85 (8H, m, br, acetate CH₂'s), 5.64-5.86 (1H, m, NH), 7.65 (1H, d, J8.0, aromatic H6), 7.84 (1H, d, J8.0, aromatic H5), 7.98 (1H, s, aromatic H3); δ_{C} (50 MHz, D₂O) 42.4 (NC═OCH₂), 47.8 (2×NCH₂), 49.3 (2×NCH₂), 52.1 (2×NCH₂), 53.2 (2×NCH₂), 55.0 (3×acetate CH₂'s), 112.9-130.1 (aromatic and CF3's), 163.1 (NC═O), 169.1 (C═O), 174.5 (2×C═O); δ_{F} (188 MHz, D₂O) −63.1 (p-CF₃), −61.8 (o-CF₃); m/z (electrospray, ES⁺) 616.3 (80%, [M+H]⁺), 638.3 (100% [M+Na]⁺). Found [M+H]⁺, 616.2200. C₂₄H₃₂O₇N₅F₆ requires [M+H]⁺, 616.2206.

### [EuL¹]

Europium (III) chloride hexahydrate (0.018 g, 0.049 mmol) and ligand L¹ (0.030 g, 0.049 mmol) were dissolved in purified water (5 ml) and the pH adjusted to 5.5 using sodium hydroxide solution. The stirred solution was heated at reflux for 24 hours. The solution was brought up to pH 10 and then centrifuged to remove the precipitated Eu³⁺ salts. The pH was then adjusted back to 5.5 and the solvent removed on the freeze drier to give white crystals.

Purification was by column chromatography through a plug of alumina (20% MeOH, 80% DCM), the solvent was then removed under reduced pressure and the resulting solid taken into water and freeze dried to yield an amorphous white solid (35 mg, 96%). δ_{H} (200 MHz, D₂O) [partial assignment] 10-(−18) (8H, m, br, acetate CH₂'s), −8 (4H, s, ring-H_{eq}), −5 (2H, s, ring-Hₐₓ), −4 (2H, d, ring-Hₐₓ), 0.5 (2H, s, ring-H_{eq}), 0.8 (2H, d, ring-H_{eq}), 8 (1H, s, Ar), 10 (1H, d, Ar), 11 (1H, s, Ar), 29 (2H, br, ring-Hₐₓ), 30 (1H, s, ring-Hₐₓ), 34 (1H, s, ring-Hₐₓ); δ_{F} (188 MHz, D₂O) −64.1 (minor isomer CF₃), −63.1 (minor isomer CF₃), −62.8 (minor isomer CF₃), −62.5 (major isomer 2×CF₃), −61.2 (minor isomer CF₃); m/z (electrospray, ES⁻) 764.5 (63%, [M]). Found: [M+Na]⁻ 788.0987. C₂₄H₂₈F₆N₅O₇¹⁵³Eu²³Na requires [M+Na]⁻, 786.0983.

The following complexes were made in a similar way:

### [TbL¹]

δ_{H} (200 MHz, D₂O) [Partial assignment] −149 (8H, s, acetate CH₂'s), −131, −114, −99, −74, −65 (4H, d, ring-H_{eq}), −46 (4H, d, ring-Hₐₓ), −27, 14, 17, 23, 30, 32, 120, 130, 185, 205, 240 (4H, m, br, ring-Hₐₓ); δ_{F} (188 MHz, D₂O) −75.1 (major isomer p-CF₃), −63.2 (minor isomer CF₃), −59.1 (minor isomer CF₃), −56.9 (minor isomer CF₃), −52.3 (minor isomer CF₃), −51.2 (major isomer o-CF₃); m/z (electrospray, ES⁻) 770 (100%, [M−H]⁻). Found: [M+Na]⁻, 794.1038. C₂₄H₂₈F₆N₅O₇¹⁵⁹Tb²³Na requires [M+Na]⁻, 794.1044.

### [YbL¹]

δ_{H} (400 MHz, D₂O) [partial assignment] −72, −70, −62, −47, −42, −24, −23, −18, −6, 10, 15, 17, 19, 23, 27, 30, 37, 110 (2H, br, ring-Hₐₓ), 117 (1H, s, ring-Hₐₓ), 129 (1H, s, ring-Hₐₓ); δ_{F} (188 MHz, D₂O) −65.4 (o-CF₃), −59.6 (p-CF₃); m/z (electrospray, ES⁻) 785.2 (100%, [M]⁻). Found: [M−H]⁻ 781.1167. C₂₄H₂₇F₆N₅O₇¹⁷⁰Yb requires [M−H]⁻, 781.1167.

### [DyL¹]

δ_{F} (188 MHz, D₂O) −80.2 (major isomer p-CF₃), −70.2 (minor isomer CF₃), −65.3 (major isomer o-CF₃), −63.4 (minor isomer CF₃), −61.9 (minor isomer CF₃), −57.1 (minor isomer CF₃) −47.3 (minor isomer CF₃); m/z (electrospray, ES⁻) 771 (100%, [M−H]⁻). Found: [M−H]⁻, 771.1072. C₂₄H₂₇F₆N₅O₇¹⁶⁰Dy requires [M−H]⁻, 771.1072.

### [TmL¹]

δ_{F} (188 MHz, D₂O) −79.6 (o-CF₃), −55.6 (p-CF₃); m/z (electrospray, ES⁻) 781 (100%, [M−H]⁻). Found: [M−H]⁻, 780.4255. C₂₄H₂₇F₆N₅O₇¹⁶⁹Tm requires [M−H]⁻, 780.4251.

### [YL¹]

δ_{H} (400 MHz, D₂O) [partial assignment] 2.36-3.85 (12H, m, br, cyclen ring), 3.11-3.85 (8H, m, br, acetate CH₂'s), 3.81 (2H, d, ring-Hₐₓ), 4.01 (2H, d, ring-Hₐₓ), 7.79 (1H, d, J8.0, aromatic H6), 7.97 (1H, d, J8.0, aromatic H5), 8.11 (1H, s, aromatic H3); δ_{F} (188 MHz, D₂O) −63.3 (p-CF₃), −61.5 (o-CF₃); m/z (electrospray, ES⁻) 700 (100%, [M−H]⁻). Found: [M−H]⁻, 700.0874. C₂₄H₂₇F₆N₅O₇⁸⁹Y requires [M−H]⁻, 700.0878.

### N-2-Chloroethyl-2′,5′bis(trifluoromethyl)benzenesulfonamide

2,5-Bis(trifluoromethyl)benzenesulfonyl chloride (2 g, 6.39 mmol) was added to a solution of ethanolamine (0.20 g, 3.19 mmol) and pyridine (0.62 g, 7.67 mmol) in dichloromethane (15 cm³) while maintaining the temperature below −10° C. The reaction mixture was kept at 4° C. overnight and then poured onto ice. The product was extracted using dichloromethane (15 cm³), washed with water (3×20 cm³), dried (K₂CO₃), filtered and solvent removed under reduced pressure to yield a white solid that was purified using column chromatography on silica, (DCM, R_{f}=0.5) to give a colourless solid (0.5 g, 44%), m.p. 65-67° C., m/z (ES+): 355, δ_{H} (CDCl₃, 200 MHz): 3.41 (m, 2H, CH₂NH), 3.59 (t, J=6 Hz, 2H, CH₂Cl), 5.29 (t, J=6 Hz, 1H, NH), 8.00 (m, 2H, Ar, C3 and C4), 8.47 (s, 1H, Ar C6), δ_{C}(CDCl₃, 50.3 MHz): 43.52 (CH₂NH), 45.23 (CH₂Cl), 119.82 [q, ¹J_{CF} 18, C-2(CF₃)], 125.29 [q, ²J_{CF} 18, (C-5(CF3)], 128.50 (Ar CH), 128.61 (Ar CH), 129.92 (Ar CH), 133.07 (q, ¹J_{CF} 162 Hz, CF₃), 140.64 (Ar C) . . . δ_{F} (CDCl₃, 188 MHz): −58.2 (o-F), −63.7 (m-F). Found: C, 33.66%; H, 2.22%; N, 3.45%. C₁₀H₈NSO₂F₆Cl requires: C, 33.76%; H, 2.25%; N, 3.39%.

### 1-(2′,5′-Bis(trifluoromethyl)benzenesulfonamidoethyl)-4,7,10,tris(tert-butoxycarbonylmethyl)-1,4,7,10-tetraazacyclododecane (tri ^{t}butyl ester of H₄L²)

To N-2-chloroethyl-2,5-bis(trifluoromethyl)benzene sulfonamide, (0.15 g, 0.42 mmol) was added to a solution of 1,4,7-tris(tert-butoxycarbonylmethyl)-1,4,7,10-tetra azacyclo dodecane, 1, (0.22 g, 0.42 mmol) and K₂CO₃ (0.09 g, 0.63 g) in acetonitrile (10 cm³) and the mixture was heated at reflux overnight. Inorganic solids were filtered off, solvent was removed under reduced pressure and the tris-ester N-alkylated product was purified using column chromatography on silica, (5% MeOH/DCM R_{f} 0.3) to give an oily material, (0.25 g, 79%). m/z (ES+): 834 (M⁺), 856 [(M+Na)⁺], 872 [(M+K)⁺] . . . δ_{H} (CDCl₃, 200 MHz: 1.46 (s, 27H, t-Bu)), 2.43 (br, 16H, ring CH₂N), 2.55 (m, 6H, NCH₂CO), 3.09 (br, 4H, NCH₂), 3.31 (s, br, 1H, NH), 7.57 (d, 1H, J=8 Hz, Ar ring C4), 7.77 (d, 1H, J=8 Hz, Ar ring C3), 8.49 (s, 1H, Ar ring C6). δ_{C}(CDCl₃, 50.3 MHz): 28.27, 28.43 [C(CH₃)], 43.06 (CH₂NH), 52.22, 53.60, 55.61, 56.93, 58.52 (CH2N), 56.41, 57.22, 57.52 (CH2CO), 81.93[C(CH₃)], 119.80 [q, ²J_{CF} 18, C-2 (CF₃)], 125.27[q, ²J_{CF} 18, (C-5 (CF₃)], 128.51 (Ar CH), 128.63 (Ar CH), 129.92 (Ar CH), 133.07 (q, ¹J_{CF} 163 Hz, CF₃), 140.64 (Ar C1) 171.4, 171.6 (C═O). δ_{F}(CDCl₃, 188 MHz): −58.4 (o-F), −63.5 (m-F).

### H₆L² (CF₃CO₂⁻)₂

Ligand L² (H₄L²), (as its tri ^{t}butyl ester) (0.25 g, 3.0 mmol) was dissolved in dichloromethane (2 mL) and treated with TFA (3 mL) and the solution stirred at room temperature overnight. Solvent was removed under reduced pressure and residual TFA was removed by the stepwise addition of DCM (3×10 cm³), removing solvent under reduced pressure each time to give the ligand as its trifluoroacetate salt, as a glassy solid. M.p. 142-146° C. m/z (ES−): 666 (M⁻). δ_{H} (CD₃OD, 200 MHz): 2.9-3.8 (br m, 25H, NCH₂ and NCH₂ ring), 4.11 (br s, 2H, CH₂NH), 8.18 (br s, 2H, Ar H), 8.45 (br s, 1H. Ar H). .δ_{C} (CD₃OD, 50.3 MHz): 41.11 (CH₂NH) 42.41, 47.83, 49.21, 52.11 (CH₂N), 59.42, 60.13, 60.52 (CH₂CO), 119.81 (q, ²J_{CF} 18, C-2(CF₃)], 125.29 (q, ²J_{CF} 18, (C-5 (CF₃)], 128.52 (Ar CH), 128.61 (Ar CH), 129.91 (Ar CH), 133.06 (q, ¹J_{CF} 162, CF₃), 140.64 (Ar C1) 173.41, 174.62 (0=0). δ_{F}(D₂O, 188 MHz): −58.7 (o-F), 63.7 (m-F).

### 2-Trifluoromethylphenylsulfonylaminoethyl-2-trifluoromethylphenylsulfonate

2-Trifluoromethylbenzenesulfonyl chloride (3 g, 12.3 mmol) was added to a solution of ethanolamine (0.37 g, 6.2 mmol) and pyridine (1.16 g, 14.3 mmol) in dichloromethane (15 cm³) while maintaining the temperature below −10° C. The reaction mixture was kept at 4° C. overnight and then poured onto ice. The product was extracted using dichloromethane (15 cm³), washed with water (3×20 cm³), dried (K₂CO₃), filtered and solvent removed under reduced pressure to yield a white solid which was purified using column chromatography on silica, (DCM, R_{f}=0.3) to give a colourless solid. Yield:(1.5 g, 52%), m.p. 132-133° C., m/z (ES+): 500 (M+Na)⁺, δ_{H} (CD₃OD, 500 MHz): 3.29 (t, J=6 Hz, 2H, CH₂NH), 3.30 (s, OH), 4.11 (t, J=6 Hz, 2H, CH₂O), 4.87 (s, broad, NH), 7.75 (dd, J=7.5 Hz, Ar—C5), 7.86 (dd, J=7.5 Hz, Ar—C5′), 7.88 (dd, J=7.5 Hz, Ar—C4′), 7.89 (dd, J=7.5 Hz, Ar—C6), 7.90 (dd, J=7.5 Hz, Ar—C3), 8.00 (d, J=7.5 Hz, Ar—C3′), 8.11 (dd, J=7.5 Hz, Ar—C4), 8.20 (d, J=7.5 Hz, Ar—C6′), δ_{C}(CD₃OD, 125.6 MHz): 41.72 (CH₂NH), 59.72 (CH₂O), 122.9 [q, 1J_{CF}=275 Hz, ArCF3)], 127.7 [q, ¹J_{CF}=114 Hz, Ar′CF)], 128.4 [q, ²J_{CF}=6 Hz, C2CF3)], 128.7 [q, ²J_{CF} 6 Hz, C2′CF3)], 130.7 (ArCH), 132.18 (ArCH), 132.70 (ArCH), 132.8 (ArCH, 132.98 (ArCH), 134.11 (ArC), 134.47 (ArCH). 139.53 (ArCH), δ_{F} (CD₃OD, 188 MHz): −58.6, 58.9. Found: C, 40.24%; H, 2.75%; N, 2.92%. C₁₆H₁₃NSO₅F₆ requires: C, 40.25%; H, 2.73%; N, 2.93%.

### 1-(2-Trifluoromethylbenzenesulfonamidoethyl)-4,7,10,tris(tert-butoxycarbonylmethyl)-1,4,7,10-tetraazacyclododecane

2-(Trifluoromethylphenylsulfonylamino)ethyl(2-trifluoromethylphenylsulfonate) (0.46 g, 0.96 mmol) was added to a suspension of 1,4,7-tris(tert-butoxycarbonylmethyl)-1,4,7,10-tetraazacyclododecane, 1, (0.50 g, 0.96 mmol) and K₂CO₃ (0.26 g, 1.92 mmol) in acetonitrile (20 cm³) heated under reflux overnight. Inorganic salts were removed by filtration, solvent was removed under reduced pressure and the product was purified using column chromatography on silica, (5% MeOH/DCM R_{f}=0.3) to give an oily material, Yield: (0.45 g, 60%), m/z (ES+): 766 (M⁺), 789 [(M+Na)⁺], .δ_{H} (CDCl₃, 200 MHz): 1.46 (s, 27H, ^{t}Bu)), 2.43 (br, 16H, ring CH₂N), 2.65 (m, 6H, NCH₂CO), 3.00 (br, 4H, NCH₂), 3.41 (br s, 1H, NH), 6.62 (dd, 1H, J=8 Hz, Ar—C4), 7.69 (dd, 1H, J=8 Hz, Ar—C2), 8.49 (dd, 1H, J=8 Hz Ar—C3), 8.28 (d, 1H, J=8 Hz, ArC6), δ_{C}(CDCl₃, 50.3 MHz): 28.27, 28.43 [C(CH₃)], 43.06 (CH₂NH), 52.22, 53.60, 55.61, 56.93, 58.52 (CH₂N), 56.41, 57.22, 57.52 (CH₂CO), 81.93 [C(CH₃)], 119.80 [q, ²J_{CF}=18 Hz, C (CF₃)], 125.27, 128.5, 128.63 (Ar CH), 129.92 (Ar CH), 133.07 (q, ¹J_{CF}=163 Hz, CF₃), 140.64 (Ar C) 171.4, 171.6 (C═O). δ_{F} (CDCl₃, 188 MHz): −57.9.

### H₆L^{2′} (CF₃CO₂⁻)₂

The tris-t-butyl ester (0.45 g, 0.6 mmol) was dissolved in dichloromethane (2 mL) and treated with TFA (3 mL) and the solution stirred at room temperature overnight. Solvent was removed under reduced pressure and residual TFA was removed by the stepwise addition of DCM (3×10 cm³), removing solvent under reduced pressure each time to give the ligand as its trifluoroacetate salt, as a glassy solid, in quantitative yield. m.p. 142-146° C. m/z (ES−): 598 (M⁻). δ_{H} (D₂O, 200 MHz): 2.98 (br m, 8H, NCH₂ and NCH₂ ring), 3.26 (br m, 12H, NCH₂ring), 3.48 (br s, 4H, NCH₂CO), 3.96 (br s, 2H, NCH₂CO), 7.71 (br s, 2H, Ar H), 7.85 (br s, 1H. Ar H) 7.97 (br s, 1H. Ar H) . . . δ_{C}(CD₃OD, 50.3 MHz): 41.11 (CH₂NH) 42.41, 47.83, 49.21, 52.11 (CH₂N), 59.42, 60.13, 60.52 (CH₂CO), 119.81 (q, ²J_{CF} 18, C(CF₃)), 125.29, 128.52, 128.61, 129.91 (Ar CH), 133.06 (q, ¹J_{CF} 162, CF₃), 140.64 (Ar C1) 173.41, 174.62 (C═O). .δ_{F}(D₂O, 188 MHz): −58.3.

### Synthesis of Lanthanide (III) Complexes:

The ligand H₆L²(CF₃CO₂)₂ and an equimolar quantity of LnCl₃(H₂O)₆ (1:1) (Ln=Eu, Tb, Dy or Yb) were taken into the minimum volume of Purite water (<1 mL) and the pH was adjusted to 5.5. The solution was heated at 90*C for 24 hrs. The reaction mixture was cooled to room temperature and pH was adjusted to 10. The resulting white precipitate was filtered off, and the solution pH was adjusted to 5.5. The solvent was removed by lyophilisation and the residue was extracted from the residue using 10% MeOH/DCM to yield a colourless or pale cream solid. For each complex, excellent agreement was observed between found and calculated isotope patterns for their negative ion electrospray mass spectra, using solutions presented in methanol.

[Eu.L²]: m/z (MeOH, ES−): 812, 814. Found: 812.0842; C₂₄H₂₉O₈N₅F₆SEu requires: 812.0845; δ_{F} (D₂O, 188 MHz) pH 5.5: −65.19, −63.90, −58.97, −53.4 (1:1; 1; 1); pH 10.2: −65.15, −53.36, pH 4.2: −63.87, −58.93.

[Tb.L²]: m/z (MeOH, ES−): 820, 821. Found 820.0887; C₂₄H₂₉O₈N₅F₆STb requires: 820.0890; δ_{F}(D₂O, 188 MHz): pH 5.5: br −45.5, br −61.5, −157.5.

pH 10: −44.87, −159.59; pH 3.5: −63.9, −58.89

[Dy.L²]: m/z (ES−): 822, 823, 824, 825, 826. Found: 822.0913; C₂₄H₂₉O₈N₅F₆SDy requires: 822.0916; δ_{F}(D₂O, 188 MHz): pH 6.0: −38.95, br 59.1, 159.18; pH 10: −39.39, −158.41; pH 3.5: −59.13, −64.22.

[Yb.L²] (exists as mixture of two major isomers): m/z (ES−): 831, 832, 833, 834, 835, 836, 837, 838. Found: 833.1010; C₂₄H₂₉O₈N₅F₆Syb requires: 822.1010; .δ_{F}(D₂₀, 188 MHz): pH 6.0: br −22.88, br 39.66, −61.11, −65.07, br −70.3; pH10: br −22.73, br −39.82, br −70.63; pH 3.5: br −59.05 (minor), br −61.77, br 64.29 (minor), br −65.77.

The ligand H₆L^{2′} (CF₃CO₂)₂ was dissolved in water (1 ml) and treated with excess anion exchange resin (Dowex 1×8 Cl, 200-400 mesh), and filtered. An equimolar quantity of LnCl₃(H₂O)₆ (1:1) (Ln=Ho, Tm, Eu) was added dissolved in the minimum volume of Purite water (total volume <1.5 mL) and the pH adjusted to 5.5. The solution was heated at 90° C. for 24 h. The reaction mixture was cooled to room temperature and pH was adjusted to 10. The resulting fine white precipitate was filtered off, and the solution pH was re-adjusted to 5.5. The solvent was removed by lyophilisation and the complex was extracted from the residue using 10% MeOH/DCM to yield a colourless or pale cream solid. For each complex, excellent agreement was observed between found and calculated isotope patterns in their negative ion electrospray mass spectra, using solutions presented in methanol.

[Eu.L^{2′}]: m/z (MeOH, ES⁻): 746 (M⁻). Found: 746.2031; C₂₃H₃₁O₈N₅F₃SEu requires: 746.2032; δ_{F} (D₂O, 188 MHz) pH 5.8: −53.2, −58.3 (1:1); pH 10.2: −53.2; pH 4.2: −58.3.

[Ho.L^{2′}]: m/z (MeOH, ES⁻):782 (M⁻). Found 782.1041; C₂₃H₃₁O₈N₃F₃SHoNa requires: 782.1043; δ_{F} (D₂O, 188 MHz): pH 5.8: −57.1, −96.4; pH 10: −96.4; pH 4.0: −57.1.

### 3-Trifluoromethyl-4-nitro-methoxyethoxymethylbenzene

3-Trifluoromethyl-4-nitro-phenol (1.0 g, 4.9 mmol) and sodium hydride (0.11, 4.9 mmol) were taken to anhydrous THF (30 ml). MEM chloride (0.6, 4.9 mmol) was added to the solution keeping the temperature below 0° C. using iso-propanol and dry ice. The solution was warmed to room temperature and stirred for 1 h. Inorganic residues were filtered off; solvent was removed under reduced pressure to leave a pale yellow residue. The product was isolated following purification using column chromatography on silica, (DCM/2% MeOH, R_{f}=0.4, DCM) to give a pale yellow oil, (1.2 g, 85%), m/z (ES⁺): 318 [(M+Na)⁺]. δ_{H} (CDCl₃, 200 MHz): 3.35 (s, 3H, O—CH₃), 3.53 (t, J=6 Hz, 2H, O—CH₂), 3.82 (t, J=6 Hz, 2H, O—CH₂), 5.36 (s, 2H, O—CH₂—O), 7.32 (dd, J H—H(o)=8 Hz, J H—H(m)=4 Hz, 1H, Ar H ortho), 7.43 (d, J_{H—H(m)}=4 Hz, 1H, Ar H ortho), 7.96 (d, J_{H—H(o)}=8 Hz, 1H, Ar H meta). δ_{C} (CDCl₃, 50.3 MHz): 58.85 (O—CH₃), 68.54 (O—CH₂), 71.47 (O—CH₂), 93.75 (O—CH₂—O), 116.44 (q, ²J_{CF}=18 Hz, C(CF₂)], 118.91 (Ar CH), 119.24 (Ar C), 125.64 (q, ¹J_{CF}=162 Hz, CF₂), 127.95 (Ar CH), 141.61 (Ar C), 160.61 (Ar CH). δ_{F}(CDCl₂, 188 MHz): 60.59 (s).

### 3-Trifluoromethyl-4-amino-methoxyethoxymethylbenzene

3-Trifluoromethyl-4-nitro-methoxyethoxymethybenzene. (1.2 g, 4.1 mmol) was dissolved in ethanol and a catalytic amount of Pd(OH)₂/C was added and the mixture stirred at room temperature under hydrogen (40 psi) for 24 hrs. Catalyst was filtered and solvent removed under reduced pressure to give a pale yellow oil. m/z (ES⁺):288 [(M+Na)⁺], δ_{H} (CDCl₃, 200 MHz): 3.30 (s, 3H, O—CH₂), 3.50 (t, J=6 Hz, 2H, O—CH₂), 3.75 (t, J=6 Hz, 2H, O—CH₂), 5.15 (s, 2H, O—CH₂—O), 6.77 (d, J_{H—H(o)}=8 Hz, 1H, Ar H ortho), 7.00 (d, J_{H—H(o)}=8 Hz, 1H, Ar H meta), 7.15 (s, 1H, Ar H ortho). δ_{C} (CDCl₃, 50.3 Hz): 59.18 (O—CH₂), 67.78 (O—CH₂), 71.76 (O—CH₂), 94.66 (O—CH₂—O), 115.18 (q, ²J_{CF}=18 Hz, CCF₂), 119.21 (Ar CH), 125.64 (q, ¹J_{CF}=162 Hz, CF₂), 122.22 (Ar CH), 123.29 (Ar C), 138.72 (Ar C), 149.74 (Ar CH). δ_{F}(CDCl₂, 188 MHz): 60.59 (s).

### 3-Trifluoromethyl-4-(2-chlorocarbamoylmethyl)methoxyethoxymethylbenzene

3-Trifluoromethyl-4-amino-methoxyethoxymethylbenzene (0.5 g, 0.2 mmol), and N-hydroxysuccinimidyl chloroacetate (0.36 g, 0.2 mmol) were dissolved in dichloromethane (10 cm³), and stirred at room temperature overnight. Solvent was removed and the product purified using column chromatography on silica, (DCM/MeOH, R_{f}=0.6, 5% MeOH/DCM), followed by recrystallisation from hexane/EtOAc to give a white solid, (0.25 g, 52%), m.p. 72-74° C. Found C, 45.76%, H, 4.63%, N, 4.21%. C₁₃H₁₅NO₄F₃Cl requires C, 45.72%, H, 4.43%, N, 4.13%. m/z (ES⁺):341 (M⁺), 364 [(M+Na)⁺], δ_{H} (CDCl₃, 400 MHz): 3.37 (s, 3H, O—CH₃), 3.55 (t, J=4.8 Hz, 2H, O—CH₂), 3.81 (t, J=4.8 Hz, 2H, O—CH₂), 4.22 (s, 2H, CH₂Cl), 5.28 (s, 2H, O—CH₂—O), 7.27 (d, J_{H—H(o)}=8 Hz, 1H, Ar H ortho), 7.34 (s, 1H, Ar H ortho). 7.96 (d, J_{H—H(o)}=8 Hz, 1H, Ar H meta), 8.57 (br s, 1H, NH). δ_{C}(CDCl₃, 100.6 Hz): 43.10 (CH2Cl), 59.25 (O—CH₃), 68.09 (O—CH₂), 71.68 (O—CH₂), 93.82 (O—CH₂—O), 114.65 (q, ²J_{CF}=18 Hz, CCF₃), 120.37 (Ar CH), 122.97 (q, ¹J_{CF}=162 Hz, CF₃), 126.89 (Ar CH), 127.96 (Ar C), 154.82 (Ar C), 164.63 (Ar CH), 178.54 (C═O)], δ_{F}(CDCl₃, 188 MHz): 61.5 (s).

### Tris-t-butyl Ester of MEM Protected L^{3a}.

3-Trifluoromethyl-4-(2-chlorocarbamoylmethyl)-methoxyethoxymethylbenzene, (0.10 g, 0.3 mmol) was added to a solution of 1,4,7-tris(t-butoxycarbonylmethyl)-1,4,7,10-tetraazacyclododecane(1) (0.16 g, 0.31 mmol) and K₂CO₃ (0.04 g, 0.63 mmol) in acetonitrile (10 cm³) and the mixture was boiled under reflux overnight. Inorganic solids were filtered off, solvent was removed and the product was purified using column chromatography on silica, 5% MeOH/DCM R_{f}=0.2) to give a clear oily product, (0.10 g, 79%). m/z (ES⁺): 821 (M⁺), 844 [(M+Na)⁺], δ_{H} (CDCl₃, 400 MHz): 1.42 (S, 27H, t-Bu)), 2.13-2.62 (br, 16H, ring CH₂N), 2.72-3.10 (m, 6H, NCH₂CO), 3.09 (br, 4H, NCH₂), 3.31 (s, br, 1H, NH), 3.55 (s, 3H, OCH₃), 3.54 (d, J=4.8 Hz, 2H, OCH₂), 3.78 (d, J=4.8 Hz, 2H, OCH₂), 5.22 (2H, s, OCH₂O), 7.15 (d, J_{H—H(o)}=8 Hz, 1H, Ar H ortho), 7.25 (s, 1H, Ar H ortho). 7.45 (d, J_{H—H(o)}=8 Hz, 1H, Ar H meta). .δ_{C}(CDCl₃, 100.6 MHz): 28.06, 28.38 (C(CH3)), 43.06 (CH₂NH), 52.21, 53.61, 55.62, 56.90, 58.51 (CH₂N), 56.43, 57.21, 57.53 (CH₂CO), 59.25 (O—CH₃), 68.09 (O—CH₂), 71.68 (O—CH₂), 81.94 (CCH₃), 93.82 (O—CH₂—O), 114.65 (q, ²J_{CF}=18 Hz, C(CF₃)], 120.37 (Ar CH), 122.97 (q, ¹J_{CF}=162 Hz, CF₃), 126.89 (Ar CH), 127.96 (Ar C), 154.82 (Ar C), 164.63 (Ar CH), 171.4, 171.6 (C═O), 178.54 (C═O), δ_{F}(CDCl₃, 376.3 MHz): 61.14 (s).

### H₅L^{3a} (CF₃CO₂)₂

Ligand L³ (0.10 g, 0.12 mmol) was dissolved in dichloromethane (2 cm³) and TFA (3 cm³) was added and the mixture was stirred at room temperature overnight. Solvent was removed under reduced pressure. Traces of TFA were removed by the successive addition of DCM (3×10 cm³) removing solvent each time under reduced pressure to give the trifluoroacetate salt, as a glassy solid,

m.p. 122-124° C. m/z (ES⁻): 563 (M⁻). δ_{H} (CD₃OD, 400 MHz): 2.9-3.8 (br m, 25H, NCH₂ and NCH₂ ring), 4.11 (br s, 2H, CH₂NH), 7.75 (d, J_{H—H(o)}=8 Hz, 1H, Ar H ortho), 7.95 (s, 1H, Ar H ortho). 8.31 (d, J_{H—H(o)}=8 Hz, 1H, Ar H meta). .δ._{C}(CD₃OD, 50.3 MHz): 41.11 (CH₂NH) 42.41, 47.83, 49.21, 52.11 (CH₂N), 59.422, 60.13, 60.52 (CH₂CO), 119.80 (q, ²J_{CF}=18 Hz, C (CF3), 121.32 (Ar CH), 124.97 (q, ¹J_{CF}=162 Hz, CF₃) 127.89 (Ar CH), 129.96 (Ar C), 130.82 (Ar C), 168.63 (Ar CH), 178.54 (C═O). δ_{F} (CDCl₃, 376.3 MHz): −61.54 (s). δ_{F} (CD₃OD, 376.3 MHz): −62.8 (s).

The ligand H₃L^{3a} (CF₃CO₂)₂ and an equimolar quantity of LnCl₃(H₂O)₆ (1:1) (Ln=Eu, Tb, Dy or Yb) were taken into the minimum volume of Purite water (<1 mL) and the pH was adjusted to 5.5. The solution was heated at 90° C. for 24 h. The reaction mixture was cooled to room temperature and pH was adjusted to 10. The resulting white precipitate was filtered off, and the solution pH was adjusted to 5.5. The solvent was removed by lyophilisation and the complex was extracted from the residue using 10% MeOH/DCM to yield a colourless or pale cream solid. For each complex, excellent agreement was observed between found and calculated isotope patterns for their positive ion electrospray mass spectra, using solutions presented in methanol. Fluorine-19 NMR shifts (±0.3 ppm) and linewidths (±4 Hz) for the complexes listed were invariant over the pH range 4 to 9.

[Tm.L^{3a}]: m/z (MeOH, ES⁺): 752. Found: 752.1200; C₂₃H₂₉O₈N₅F₃NaTm requires: 752.1202; δ_{F}(D₂O, 188 MHz) pH 7.5: −78.1 (ω_{1/2}=36 Hz), −89.2 (minor isomer 9%).

[Tb.L^{3a}]: m/z (MeOH, ES⁺): 720, 742. Found 742.1115; C₂₃H₂₉O₈N₅F₃NaTb requires: 742.1114; δ_{F}(D₂O, 188 MHz): pH 7.4: br −52.5 (ω_{1/2}=52 Hz)), br −40.6 (minor isomer 12%).

[Ho.L^{3a}]: m/z (ES⁺): 748. Found: 748.1169; C₂₃H₂₉O₈N₅F₃NaHo requires: 748.1164; δ_{F} (D₂O, 188 MHz): pH 7.8: br −57.9 (ω_{1/2}=31 Hz), br −49.0 (minor isomer 12%).

[Dy.L^{3a}]: m/z (ES⁺): 745. Found: 747.1165; C₂₃H₂₉O₈N₅F₃NaDy requires 747.1158; . . . δ_{F} (D₂O, 188 MHz): pD 7.5: br −66.7 (ν_{1/2}=64 Hz), br −43.8 (minor 20%).

[Gd.L^{3a}]: m/z (ES⁺): 742. Found: 738.1101; C₂₃H₂₉O₈N₅F₃NaGd requires: 738.1087; δ_{F} (D₂O, 376 MHz): −61 ppm (ν_{1/2}=3,500 Hz).

H₅L^{3b} and L^{3c} may be made from L^{3a} according to standard conditions know to the skilled person, for example L^{3b} may be made from L^{3a} using the procedure of R. P. Mason et al. (vide supra) or using the method Y. Urano et al (J. Am. Chem. Soc., 2007, 129, 3918); L^{3c} may be made by acylating L^{3a} in aqueous dioxan under phase transfer conditions.

### Ligands L⁴ and L⁵

### 2-Chloro-N-(4-nitro-2-trifluoromethyl)-ethanamide

Chloroacetylchloride (1.95 ml, 24.5 mmol) was added dropwise to a stirred solution of 4-nitro-2-trifluoromethyl aniline (2.01 g, 9.8 mmol), 4-dimethylaminoipyridine (10 mg) and triethylamine (1.70 ml, 12.1 mmol) in dry THF (35 ml) at 0° C. The reaction mixture was allowed to warm to room temperature and stirred for 48 h. CH₂Cl₂ (30 ml) was added and the resulting white precipitate dissolved in H₂O (30 ml). The organic layer was washed with HCl_{(aq)} (0.1 M, 30 ml) followed by H₂O (3×20 ml) then dried over K₂CO₃. After filtration the solvent was removed to yield a brown oil that was purified by column chromatography over silica gel eluting with CH₂Cl₂:hexane (50:50 then 80:20 and finally 100% CH₂Cl₂) giving a light yellow crystalline solid (1.35 g, 49%), m.p. 62-64° C. δ_{H} (200 MHz, CDCl₃): 4.28 (2H, s, CH₂Cl), 8.44 (1H, d, J=9.0, H⁶), 8.54 (1H, s, H³), 8.69 (1H, d, J=9.0, H⁵), 9.08 (1H, br, s, NH); δ_{C} (50 MHz, CDCl₃) 43.09 (CH₂Cl), 122.59, 122.70, 123.05, 128.57, 140.15, 143.75 (Ar), 164.68 (0=0); δ_{F} (188 MHz, CDCl₃) −61.71 (CF₃); m/z (ESMS⁻) 281.3 [M−H]⁻. Found C, 38.3; H, 2.12; N, 9.80%. C₉H₆N₂O₃F₃Cl requires C, 38.2; H, 2.14; N, 9.91%.

### 10-[(4-Nitro-2-trifluoromethyl(phenyl))carbamoylmethyl]-1,4,7-tris(tert-butoxycarbonylmethyl)-1,4,7,10-tetraazacyclododecane

To a solution of 1,4,7-tris(tert-butoxycarbonylmethyl)-1,4,7,10-tetraazacyclododecane (330 mg, 0.64 mmol) and 2-chloro-N-(4-nitro-2-trifluoromethyl)-ethanamide (200 mg, 0.71 mmol) in dry CH₃CN (20 ml) under argon, was added K₂CO₃ (106 mg, 0.77 mmol) and KI (5 mg, cat.). The mixture was boiled under reflux for 24 h. After filtration, the residue was washed with CH₂Cl₂ (2×30 ml) and solvent removed under reduced pressure to give a brown oil which was filtered through a layer of silica gel, washed first with diethyl ether and then with 20% MeOH/CH₂Cl₂. Removal of solvent under reduced pressure afforded a pale brown oil (402 mg, 83%). δ_{H} (200 MHz, CDCl₃) 1.37 (27H, br, s, CH₃), 2.15-3.80 (24H, br, CH₂ ring and CH₂CO), 8.11 (1H, d, J=8.5, aromatic H⁶), 8.25 (1H, dd, J=8.5, 2.4, aromatic H⁵), 8.48 (1H, d, J=2.5 Hz, aromatic H³); δ_{F} (188 MHz, CDCl₃) 61.10 (CF₃); m/z (ESMS⁺) 783.3 [M+Na]⁺.

### 10-[(4-Amino-2-trifluoromethyl(phenyl))carbamoylmethyl]-1,4,7-tris(tert-butoxycarbonylmethyl)-1,4,7,10-tetraazacyclododecane

10-[(4-Nitro-2-trifluoromethyl(phenyl))carbomoylmethyl]-1,4,7-tris(tertbutoxycarbonylmethyl)-1,4,7,10-tetraazacyclododecane (131 mg, 0.17 mmol) in MeOH (3 ml) was placed in a hydrogenation vessel. A small amount (ca. 15 mg) of Pd/C catalyst was added and the mixture was hydrogenated at 40 psi for 20 h. The Pd/C was removed by syringe filtration and the solvent removed under reduced pressure to yield the product as a brown-red solid (122 mg, 97%). δ_{F} (188 MHz, CDCl₃) −62.3 (CF₃); m/z (ESMS⁺) 769.3 [M]⁺.

### 10-[(4-Nitro-2-trifluoromethyl(phenyl)carbamoylmethyl]-1,4,7-tris(carboxymethyl)-1,4,7,10-tetraazacyclododecane, H₄L⁴

10-[(4-Nitro-2-trifluoromethyl(phenyl))carbamoylmethyl]-1,4,7-tris(tert-butoxycarbonylmethyl)-1,4,7,10-tetraazacyclododecane (310 mg, 0.41 mmol) was dissolved in CH₂Cl₂ (1 ml) and CF₃CO₂H (6 ml) was added. The solution was stirred at room temperature for 24 h. The solvent was removed under reduced pressure and the resulting solid washed with DCM (5×5 ml) removing the solvent each time under reduced pressure (KOH in trap) to give the product as trifluoroacetate salt (δ_{F} −76.1 TFA). The residue was dissolved in water (5 mL) and stirred overnight with anion exchange resin (DOWEX 1X8 200-400 MESH Cl, pre-treated with 1M HCl) in Purite H₂O to give the chloride salt. After filtration the water was freeze dried to yield the product as a light yellow powder (113 mg, 47%). δ_{H} (200 MHz, D₂O) 2.60-4.20 (24H, br, CH₂ ring and CH₂CO), 7.84 (1H, d, J=8.5, aromatic H⁶), 8.36 (1H, d, J=8.5, aromatic H⁵), 8.51 (1H, br s, aromatic H³); δ_{C} (126 MHz, D₂O) 46.83, 48.55, 51.13, 54.73, 79.91, 122.99, 128.04, 129.379; δ_{F} (188 MHz, D₂O) −61.75 (CF₃); m/z (ESMS⁺) 593.3 [M+H]⁺, 615.3 [M+Na]⁺, 631.2 [M+K]⁺; (ESMS⁻⁾ 629.1 [M+Cl]⁻. Found 631.1739; O₂₃H₃₁O₉N₆F₃K requires 631.1736. found 655.1403; C₂₃H₃₁O₉N₆F₃Cu requires 655.1395.

### [HoL⁴]

The ligand H⁴L⁴ (30.5 mg, 0.051 mmol) and Ho(III) Cl₃ (15.9 mg, 0.059 mmol) were dissolved in water (3 ml) and the pH adjusted to 5.5 using KOH _{(aq)}. The reaction was left stirring overnight under reflux. After cooling to room temperature, the solution pH was adjusted to pH 10 and the mixture centrifuged to remove the precipitated metal hydroxide. The supernatant was adjusted back to pH 6 with HCl_{(aq)} and freeze dried to give a pale yellow solid that was extracted into 20% MeOH in dichloromethane to give a colourless solid. δ_{H} (200 MHz, D₂O) −250 (1H, br), −248 (1H), −66 (1H), −58 (3H), −49 (1H), 55 (1H), 89 (1H), 161 (4H, br), 166 (1H); δ_{F} (188 MHz, D₂O) (exists as mixture of isomers) pD 5.5: −55.1 (br, OF₃ major isomer, 88%); m/z (ESMS⁺) 777.0 [M+Na]⁺, 793.0 [M+K]⁺. Found 777.1067; C₂₃H₂₈O₉N₆F₃HoNa requires 777.1065. found 793.0816; C₂₃H₂₈O₉N₆F₃HoK requires 793.0805.

### [DyL⁴]

δ_{F} (188 MHz, D₂O) −58.3 (br, CF₃ major isomer), −66.5 (minor); m/z (ESMS⁺) 776.0 [M+Na]⁺. Found 776.1064; C₂₃H₂₈O₉N₆F₃DyNa requires 776.1054.

### 10-[(4-Amino-2-trifluoromethyl(phenyl)carbamoylmethyl]-1,4,7-tris(carboxymethyl)-1,4,7,10-tetraazacyclododecane, H₃L⁵

This was prepared from the tris-t-butyl ester using TFA, as described for L⁴ above.

δ_{H} (200 MHz, D₂O) 2.70-4.30 (16H, br, cyclen ring), 4.60-4.90 (8H, m, br, acetate CH₂'s), 7.64 (1H, d, J=4, aromatic H⁶), 7.71 (1H, d, J=4.2, aromatic H⁵), 7.75 (1H, s, aromatic H³); δ_{C} (126 MHz, D₂O) 27.31, 29.68, 49.95, 54.03, 121.65, 127.60, 131.57, 171.15; δ_{F} (188 MHz, D₂O, pD 5.4) −62.00 (CF₃); m/z (ESMS⁻) 599.4 [M+Cl]⁻.

The following complexes were prepared as described for the complexes of L⁴.

### [TbL⁵]

δ_{H} (200 MHz, D₂O) −400 (2H), −370 (2H), −348 (1H), −140 (1H), −126 (1H), −110 (1H), −98 (2H), −94 (1H), −73 (1H), −(1H), −64 (1H), −51 (1H), −18 (1H), −15 (1H), −2 (1H), 28 (1H), 41 (1H), 50 (1H), 86 (1H), 117 (1H), 128 (1H), 140 (1H), 224 (1H, br), 230 (1H, br), 238 (1H), 252 (1H, br), 263 (1H); δ_{F} (188 MHz, D₂O) −53.0 (CF₃ major isomer), −51.9, −39.1 (minor isomers); m/z (ESMS⁺) 741.0 [M+Na]⁺; (ESMS⁻) 717.3 [M−H]⁻. Found 741.1283; C₂₃H₃₀O₇N₆F₃TbNa requires 741.1274. found 719.1462; C₂₃H₃₁O₇N₆F₃Tb requires 719.1454.

### [DyL⁵]

δ_{H} (200 MHz, D₂O) −508 (1H), −472 (1H), −439 (1H), −426 (1H), −398 (1H), −200 (1H), −155 (1H), −143 (1H), −107 (1H), −100 (1H), −99 (1H), −72 (1H), −58 (1H), −52 (1H), −45 (1H), −32 (1H), −18 (1H), −10 (1H), 10 (1H), 111 (1H), 163 (1H), 194 (1H), 215 (1H), 272 (1H, br), 316 (2H, br), 324 (1H, br); δ_{F} (188 MHz, D₂O) −66.0 (CF₃ major isomer), −46.5 (minor); m/z (ESMS⁺) 746.0 [M+Na]⁺; (ESMS⁻) 722.3 [M−H]⁻ Found: 743.1297; C₂₃H₃₀O₇N₆F₃DyNa requires 743.1290. found 724.1501; C₂₃H₃₁O₇N₆F₃Dy requires 724.1493.

1. A paramagnetic lanthanide (III) complex comprising a lanthanide (III) ion and a polydentate ligand wherein the polydentate ligand comprises one or more fluorine atoms in which the distance of at least one of the fluorine atoms to the lanthanide ion is less than 7 Å, said polydentate ligand not being a DPTA bisamide of p-CF₃-aniline.

2. The complex of claim 1 wherein the polydentate ligand comprises a macrocyclic polydentate ligand.

3. The complex of claim 1 wherein the distance is between about 4 Å and 7 Å.

4. The complex of claim 1 wherein the distance is less than 6 Å.

5. The complex of claim 1 wherein the distance is less than 5 Å.

6. The complex of claim 1 wherein the ion is selected from the group consisting of Ce, Pr, Nd, Sm, Eu, Gd, Tb, Dy, Ho, Er, Tm and Yb.

7. The complex of claim 1 wherein the ion is Dy, Tm or Tb.

8. The complex of claim 1 wherein the ion is Eu or Yb.

9. The complex of claim 1 wherein the one or more fluorine atoms are present in a trifluoromethyl group, a difluoromethyl group or a difluoromethylene group.

10. The complex of claim 2 wherein the macrocyclic ligand is a 1,4,7,10-tetraazacyclododecane.

11. The complex of claim 10 wherein three of the four nitrogen atoms of 1,4,7,10-tetraazacyclododecane are each substituted with a moiety of the formula 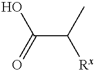
wherein R^{x} is selected from the group comprising H, straight-chain or branched C₁₋₆ alkyl or carboxy (straight-chain or branched) C₁₋₆ alkyl.

12. The complex of claim 11 wherein R^{x} is selected from the group comprising H, CH₃, CH₂CO₂⁻, CH₂CH₂CO₂⁻ or CH₂CH₂CH₂CO₂⁻.

13. The complex of claim 11 wherein R^{x} is H.

14. The complex of claim 10 wherein one of the nitrogen atoms of the 1,4,7,10-tetraazacyclododecane is substituted with 2-hydroxy-(2-trifluoromethyl)ethyl.

15. The complex of claim 10 wherein the macrocyclic ligand is of any one of the following formulae: 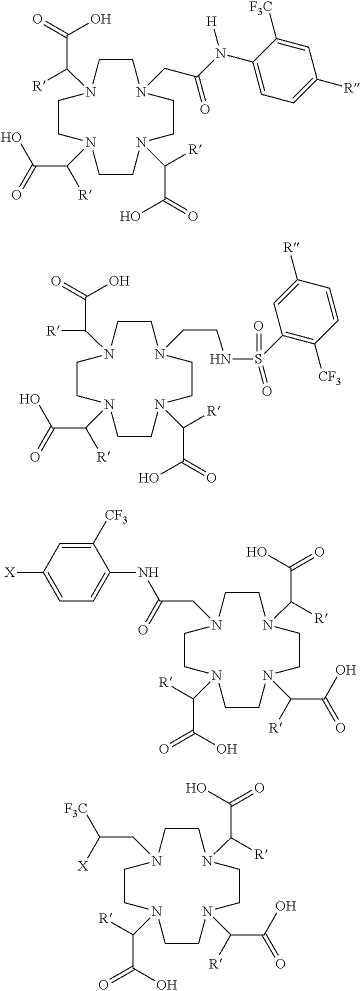
(wherein: each R′ is independently selected from the group comprising H, straight-chain or branched C₁₋₆ alkyl or carboxy (straight-chain or branched) C₁₋₆ alkyl;
R″ is H, CF₃, NO₂, CN, CF₃SO₂, NH₂, NHR^{y} or NR^{y}₂ (wherein R^{y} is selected from the group comprising H, straight-chain or branched C₁₋₆ alkyl or carboxy (straight-chain or branched) C₁₋₆ alkyl;
X is OH, OCOR″′ or
O-monosaccharide (e.g. O-β-galactose or O-glucose), NHCOR″′, NHCO-peptide, CO₂R″′, CONHR″′ or CONH-peptide; and
R″′ is alkyl or aralkyl).

16. The complex of claim 15 wherein:
each R′ is independently selected from the group comprising H, straight-chain or branched C₁₋₆ alkyl or carboxy (straight-chain or branched) C₁₋₆ alkyl;
R″ is H or CF₃;
X is OH, OCOR″′ or
O-monosaccharide (e.g. O-β-galactose or O-glucose), NHCOR″′, NHCO-peptide, CO₂R″′, CONHR″′ or CONH-peptide; and
R′″ is alkyl or aralkyl.

17. The complex of claim 15 wherein R′ is independently selected from the group H, CH₃, CH₂CO₂⁻, CH₂CH₂CO₂⁻ and CH₂CH₂CH₂CO₂⁻.

18. The complex of claim 15 wherein said monosaccharide is β-galactose or glucose.

19. The complex of claim 10 wherein the macrocyclic ligand is of any one of the following formulae: 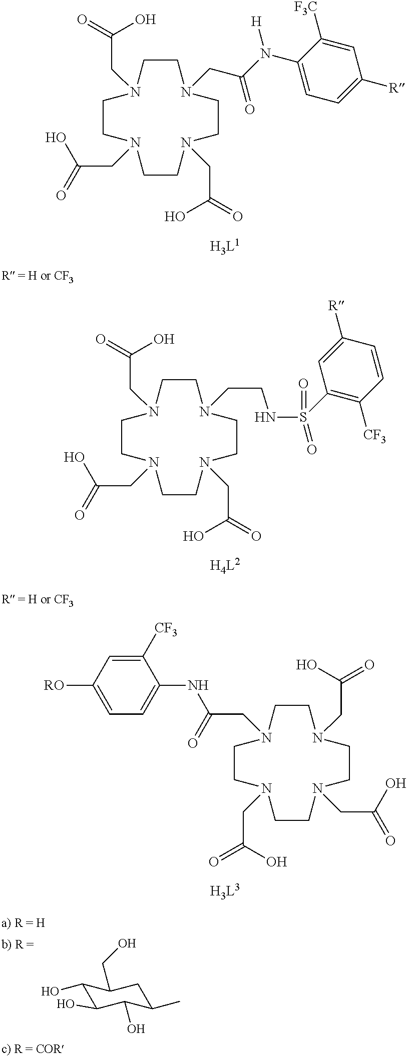
wherein R′ is optionally substituted alkyl or aralkyl.

20. The complex of claim 15 wherein R″ is CF₃.

21. The complex of claim 10 wherein the macrocyclic ligand is of the following formula:

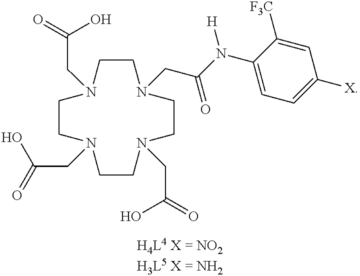

22. A macrocyclic ligand as defined in claim 15.

23. (canceled)

24. (canceled)

25. (canceled)

26. (canceled)
